(19) United States
(12) Patent Application Publication
(10) Pub. No.: US 2009/0005693 A1
(43) Pub. Date: Jan. 1, 2009

(54) Medical Imaging Methods and Apparatus for Diagnosis and Monitoring of Diseases and Uses Therefor

(75) Inventors: Raul A. Brauner, Framingham, MA (US); John Heymach, Pearland, TX (US)

Correspondence Address: WOLF GREENFIELD & SACKS, P.C., 600 ATLANTIC AVENUE, BOSTON, MA, 02210-2206, US

(73) Assignee: BioTree Systems, Inc.

(21) Appl. No.: 11/794,072

(22) PCT Filed: Dec. 22, 2005
(86) PCT No.: PCT/US2005/047081

§ 371 Date: May 2, 2008

Related U.S. Application Data
(60) Provisional application No. 60/639,196, filed on Dec. 22, 2004.

(51) Int. Cl. A61B 5/107

(52) U.S. Cl. 600/481

(57) ABSTRACT

Methods are disclosed for analyzing representations of one or more in situ structures in the body of a subject (e.g., a human subject or other animal subject) to glean information about the health of the subject. Methods are disclosed for diagnosing, staging, grading, and monitoring diseases. Methods also are disclosed for targeting treatments and screening, validating therapies based on the analysis of in situ patters (e.g., individual structural features or distributions), and monitoring the effectiveness of therapies.

### RELATED APPLICATIONS

This application claims priority under 35 U.S.C. § 119(e) from U.S. Provisional Application Ser. No. 60/639,196 with a filing date of Dec. 22, 2004 and entitled “Method and Apparatus for Analyzing Internal Body Structures” incorporated herein in its entirety by reference.

### FIELD OF THE INVENTION

Aspects of the present invention relate to analyzing images for diagnostic and therapeutic applications in animals. In particular, aspects of the invention relate to analyzing images to identify structural features in animal bodies for detecting, monitoring, and/or treating diseases, and/or for evaluating and validating new therapies.

### BACKGROUND OF THE INVENTION

A wide range of imaging methods and devices are commonly used to evaluate different anatomical and physiological conditions in a variety of medical and research environments. Tools have been developed to image body structures based on different physical properties. For example, X-rays, CT scans, MRIs, PET scans, IR analyses and other technologies have been developed to obtain images of various body structures. These tools are routinely used for diagnostic, therapeutic, and research applications. Combinations of two or more different imaging techniques are sometimes used to provide complementary information about a patient.

### SUMMARY OF THE INVENTION

Aspects of the invention relate to analyzing data obtained for in situ internal body structures in live humans and other animals. Aspects of the invention can be used to analyze data obtained from any suitable image source to identify one or more patterns associated with tubular structures of different sizes (e.g., structural patterns of blood micro-vessels). One or more parameters of a structural pattern can be used as biomarkers for different biological conditions and processes (including pathogenic conditions). Accordingly, aspects of the invention relate to disease detection, diagnosis, grading, staging, disease monitoring, monitoring the effectiveness of therapy and interventional applications based on an analysis of in situ structures to identify patterns that may be associated or correlated with a disease or other physiological condition. According to the invention, a pattern may comprise one or more different parameters. Parameters may be one or more structural features of individual tubular structures and/or one or more distribution properties (e.g., spatial distribution, spatial orientation, frequency, number, etc., or any combination thereof) of one or more tubular structures and/or one or more distribution properties (e.g., spatial distribution, spatial orientation, frequency, number, etc., or any combination thereof) of one or more individual tubular structural features within a subject or a within a region of interest in the subject, or any combination thereof. Accordingly, a vasculature pattern may include one or more structural features of an individual blood vessel (e.g., micro-vessels), a distribution of one or more blood vessels (e.g., micro-vessels) within a subject, a distribution of one or more individual blood vessel structural features (e.g., individual micro-vessel structural features), or any combination thereof. An individual blood vessel structural feature may include, but is not limited to, vessel tortuosity, curvature, branching (e.g., frequency, angle, hierarchy, etc.), diameter, direction, etc., or any change (e.g., variation or frequency) of any of these features over a predetermined length of the blood vessel being analyzed, or any combination thereof. A distribution of blood vessels or individual blood vessel structural features may include, but is not limited to, a blood vessel density, a distribution of blood vessel directions, a distribution of blood vessel diameters, a distribution of distances between blood vessels, a distribution of blood vessel spatial orientations (e.g., relative to each other), a distribution of blood vessel curvatures, a distribution of any other individual blood vessel structural features described herein, other distributions of blood vessel parameters or any combination of two or more thereof. It should be appreciated that the distribution of blood vessels or blood vessel structural features may be determined and/or analyzed for a predetermined region within a subject (e.g., a target volume of tissue within a subject) or within predetermined tissues or organs within a subject or throughout the subject. It also should be appreciated that either the absence or presence of blood vessels or of individual blood vessel structural features within a predetermined volume being analyzed may be a pattern parameter that can be used in analytical methods of the invention. It also should be appreciated that one or more pattern parameters may be monitored and/or analyzed as a function of time. Accordingly, blood vessel patterns can be used as biomarkers for different biological conditions and processes (including pathogenic conditions). Accordingly, aspects of the invention relate to disease detection, diagnosis, grading, staging, disease monitoring, monitoring the effectiveness of therapy and interventional applications based on an analysis of in situ vasculature patterns including vasculature morphology and/or architecture in live humans and other animals. In one embodiment, the in vivo density, and/or diameter distribution, and/or geometric orientation of blood vessels (e.g., micro-vessels) may be analyzed, quantified, and/or evaluated for disease detection, monitoring, and/or interventional applications. In one embodiment, the sensitivity and specificity of disease diagnosis may be enhanced by analyzing and evaluating in vivo vasculature morphology and/or architecture associated with a tissue lesion. Accordingly, aspects of the invention include detecting in vivo indicia of diseases associated with abnormal vascular structures or patterns. Other aspects include disease diagnosis, staging, grading, monitoring and prognosis, patient treatment, drug development and validation, and research applications.

One embodiment according to the present invention includes a method of analyzing geometric features of blood vessels and correlating one or more features with a biological process, condition, or disease. Accordingly, certain geometric features of blood vessels may be used as biomarkers indicative of particular biological processes, conditions, and/or diseases.

One embodiment according to the present invention includes a method of automatic disease detection or monitoring by automatically analyzing data obtained for one or more in situ, in vivo, internal body structures to determine whether they present characteristics of disease. One embodiment according to the present invention includes a method of automatically analyzing data relating to in situ vascular patterns in a live human or other animal in order to automatically detect indicia of a disease that alters the normal shape, size, and/or organization of the vasculature. Another embodiment includes a method of automatically monitoring the progression of a disease associated with changes in vascular patterns. Another embodiment includes a method of automatically monitoring the effectiveness of a therapy for a disease associated with changes in vascular patterns. Accordingly, in one embodiment, aspects of the invention relate to methods for detecting, diagnosing, staging, grading, monitoring, and treating tumors associated with angiogenesis.

One embodiment according to the present invention includes a method of detecting or evaluating a disease or condition (e.g., angiogenesis) in a live subject, the method comprising computer-implemented acts of obtaining a segmented representation of at least one in situ vasculature pattern (e.g., structure) for a live subject and gleaning the presence or absence of a disease or condition (e.g., angiogenesis) in the live subject from the in situ vasculature pattern (e.g., structure).

Another embodiment according to the present invention includes a method of detecting or evaluating a disease or condition (e.g., angiogenesis) in a live human subject, the method comprising analyzing an in situ pattern of vasculature having a diameter of less than 500 microns (e.g., at least one structural feature of at least one in situ blood vessel having a diameter of less than 500 microns) in a live human subject and determining whether the pattern (e.g., the least one in situ structural feature) is indicative of a disease or condition (e.g., angiogenesis), wherein the pattern (e.g., the structural feature) is detected using view data obtained from a CT scanner having one or more rows of detector elements.

Another embodiment according to the present invention includes a method of detecting or evaluating a disease or condition (e.g., angiogenesis) in a live subject, the method comprising analyzing an in situ pattern of vasculature having a diameter of less than 50 microns (e.g., at least one structural feature of at least one in situ blood vessel having a diameter of less than 50 microns) in a live human subject and determining whether the pattern (e.g., the least one in situ structural feature) is indicative of a disease or condition (e.g., angiogenesis), wherein the pattern (e.g., the structural feature) is detected using view data obtained from a CT scanner having one or more flat panel detectors.

Another embodiment according to the present invention includes a method of screening a subject for the presence of cancer, the method comprising computer-implemented acts of obtaining a segmented representation of an in situ vasculature pattern (e.g., of at least one in situ vasculature structure) for a live subject generating a first score related to said pattern and comparing the first score to a reference score, wherein the subject is identified as having at least one indicium of cancer if said first score is different from said reference score.

Another embodiment according to the present invention includes a method of monitoring a disease or condition (e.g., angiogenesis) in a live subject, the method comprising computer-implemented acts of processing first structure data obtained for a live subject at a first time point to identify a first structural feature or pattern relating to at least one in situ blood vessel in the live subject from the first structure data, processing second structure data obtained for the live subject at a second time point to identify a second structural feature or pattern relating to at least one in situ blood vessel in the subject from the second structure data, and comparing the first and second structural features or patterns to determine whether a vasculature feature that may be characteristic of a disease or condition (e.g., angiogenesis) changed between the first and second time points.

Another embodiment according to the present invention includes a method of identifying a target region for therapeutic treatment in a patient, the method comprising computer-implemented acts of analyzing a segmented representation to identify at least one in situ vasculature feature or pattern characteristic of a disease or condition (e.g., angiogenesis), and identifying an in situ region containing the vasculature feature or pattern characteristic of a disease or condition (e.g., angiogenesis) as a target region for therapeutic treatment. Accordingly, aspects of the invention include methods for defining the boundary for radiation therapy and image-guided therapy in a subject.

Another embodiment according to the present invention includes a method of evaluating a disease or condition (e.g., angiogenesis) in a subject, the method comprising obtaining structure data for at least one body region of a live subject, sending the structure data to a remote processor capable of processing the structure data to generate a segmented representation of at least one structural feature or pattern of at least one in situ blood vessel in the at least one body region, and analyzing the segmented representation to obtain structural information, and receiving the structural information from the remote processor, wherein the structural information provides an indication of the probability of a disease or condition (e.g., angiogenesis) in the at least one body region.

Another embodiment according to the present invention includes a method of evaluating a disease or condition (e.g., angiogenesis) in a subject, the method comprising receiving, from a remote site, structure data for at least one body region of a live subject, processing the structure data to generate a segmented representation of at least one structural feature or pattern of at least one in situ blood vessel in the at least one body region, analyzing said segmented representation to obtain structural information, and delivering the structural information to a remote site, wherein the structural information provides an indication of the probability of a disease or condition (e.g., angiogenesis) in the at least one body region.

Another embodiment according to the present invention includes a method of evaluating a disease or condition (e.g., angiogenesis) in a subject, the method comprising acts of obtaining structure data for at least one body region of a live subject, sending the structure data to a remote processor capable of generating a segmented representation of in situ vasculature at a sufficient resolution to identify structural vasculature features or patterns indicative of early an early stage disease or condition (e.g., early stage angiogenesis) and receiving the segmented representation from the remote processor.

Another embodiment according to the present invention includes a method of evaluating a disease or condition (e.g., angiogenesis) in a subject, the method comprising acts of receiving, from a remote site, structure data for at least one body region of a live subject, processing the structure data to generate a segmented representation of in situ vasculature at a sufficient resolution to identify structural vasculature features or patterns indicative of an early stage disease or condition (e.g., early stage angiogenesis), and delivering the segmented representation to a remote site.

It should be appreciated that in one embodiment, aspects of the invention include methods wherein a structural representation is sent to a remote site for analysis and an output is received from the remote site (e.g. a score, information about structural features, patterns, etc.) Similarly, in another embodiment, a structural representation may be received from a remote site, analyzed, and an output is returned to the remote site. In these embodiments, the structural representation may be a segmented representation. Alternatively, the structural representation may be segmented locally or at a remote site (e.g., before or after being sent or received).

Accordingly, aspects of the invention include methods wherein an analysis is performed on an existing representation that may be received or obtained without being generated as part of the methods. In addition, aspects of the invention include methods wherein a representation is generated based on existing structure data without the act of scanning to obtain structure data being part of the methods. In one embodiment, aspects of the invention include accessing, or receiving information about, one or more stored or archived representations, structural data sets, or combinations thereof (e.g., CT data sets or other data sets stored on a Picture Archiving and Communication System).

### BRIEF DESCRIPTION OF DRAWINGS

The accompanying drawings, are not intended to be drawn to scale. In the drawings, each identical or nearly identical component that is illustrated in various figures is represented by a like numeral. For purposes of clarity, not every component may be labeled in every drawing. In the drawings:

FIG. 1 illustrates a portion of an animal vasculature containing one or more structural features or patterns that can be selectively analyzed in accordance with one embodiment of the invention (FIG. 1A shows an example of a healthy blood vessel network; FIG. 1B shows a blood vessel network including characteristics of angiogenesis);

FIG. 2 illustrates a method for analyzing structure data relating to one or more selected internal features in an animal body in accordance with one embodiment of the invention;

FIG. 3 illustrates a method for obtaining structure data for analysis in accordance with one embodiment of the invention;

FIG. 4 illustrates a method for selectively preparing specific structure data for analysis in accordance with one embodiment of the invention;

FIG. 5 illustrates an example of a computer system that can implement one or more aspects of the invention;

FIGS. 6A, 6B and 6C illustrate transformations of an X-ray scanning process, an image reconstruction process, and the radon transform, respectively, each of which may provide structural information for analysis in accordance with one embodiment of the invention;

FIG. 7A illustrates a cylinder model of a structure in accordance with one embodiment of the invention;

FIG. 7B illustrates a configuration of a cylinder network model built from the cylinder model in FIG. 7A, in accordance with one embodiment of the invention;

FIGS. 8A and 8B illustrate a grayscale representation and a cross-section of a Gaussian density distribution for use in a model, in accordance with one embodiment of the invention;

FIG. 9 illustrates characteristic elliptical cross-sections of a cylindrical structure as it penetrates a number of scan planes;

FIG. 10 illustrates an exemplary X-ray scanning process of an elliptical object having a Gaussian density distribution;

FIG. 11 illustrates a schematic of a sinogram of the view data obtained from the X-ray scanning process illustrated in FIG. 10;

FIG. 12 illustrates a plot of a segment of a sinusoidal trace having a Gaussian profile resulting from taking the radon transform of a Gaussian density distribution;

FIG. 13 illustrates an exemplary sinogram of view data obtained from scanning an unknown structure;

FIG. 14 illustrates a schematic of a sinogram and a slope of a sinusoidal trace at a detected ridge point;

FIG. 15 illustrates a method of non-maximum suppression for eliminating ridge points identified during ridge detection, which can be used in accordance with one technique for analyzing structure data for use with embodiments of the invention; and

FIG. 16 illustrates a method of determining an orientation and/or a length of cylindrical segments by tracking corresponding locations through a plurality of slices of view data in accordance with one technique for analyzing structure data for use with embodiments of the invention.

### DETAILED DESCRIPTION

Aspects of the invention are directed to methods and devices for obtaining and/or analyzing data relating to internal structures in humans and other animal bodies. Data relating to one or more selected in situ structures may be obtained and/or analyzed to glean information about a physiological condition of an animal based on the structure (or changes in the structure). Structural information may be used for diagnostic, prognostic, therapeutic, interventional, research and/or development purposes, as well as for grading and/or staging a disease. In some embodiments, methods of the invention may involve analyzing one or more structural parameters (or one or more structural parameter changes over time) based on the in situ structure data or information. Methods of the invention may be automated. In some embodiments, methods of the invention involve detecting angiogenesis and/or changes in patterns of angiogenesis within a subject. In some embodiments, methods of the invention involve detecting micro-vasculature patterns and/or changes in micro-vasculature patterns. According to aspects of the invention, micro-vasculature consists of micro-vessels (e.g., vessels that have a diameter of less than about 1 mm, less than about 500 microns, less than about 200 microns, or smaller as described herein).

Aspects of the invention relate to business methods that may involve the marketing and/or licensing of biomarkers associated with particular biological processes, conditions, and/or diseases. In some embodiments, patterns (e.g., geometric features) of blood vessels are analyzed to identify or evaluate associations or correlations with certain biological processes, conditions, and/or diseases of interest. Pattern parameters may be identified that can be used as structural biomarkers (e.g., for clinical, diagnostic, therapeutic, and/or research applications as described herein). These biomarkers may be used to reduce the cost and increase the efficiency and sensitivity of medical and research techniques. In one embodiment, one or more biomarkers or methods of using the biomarkers may be marketed to medical or research customers or potential customers. In one embodiment, a fee-based service may be provided to medical or research organizations wherein information relating to a medical image is obtained and analyzed for the presence of one or more biomarkers and the resulting information is returned in exchange for a fee. The amount of the fee may be determined, at least in part, by the type of image information that is provided, the type and degree of analysis that is requested, and the format and timing of the analysis. It should be understood that aspects of the invention may be applicable to image information obtained from one or more of many different scanning modalities (including, but not limited to, micro CT, MDCT, rotational angiography, MRI, PACS). This information may be received from many different sources, including, but not limited to one or more of the following: medical centers, large pharmaceutical companies (e.g., in association with pre-clinical evaluations or during clinical trials), CROs (for both pre-clinical and clinical analyses), medical laboratories and practices (e.g., scanning centers), hospitals, clinics, medical centers, small biotechnology companies (e.g., in association with pre-clinical evaluations or during clinical trials), and bio-medical research organizations. The results of the analysis then may be returned to any one of these organizations. In some embodiments, the analysis results may be returned to the same entity that sent the image information. In other embodiments, the results may be returned to a different entity (e.g., the image information may be received from a scanning laboratory and the analysis may be returned to a physician). One or more steps involved with receiving the information, analyzing the structural features, processing the results and forwarding the results to a recipient may be automated. It also should be appreciated that one or more of these steps may be performed outside the United States of America. Business procedures (e.g., marketing, selling, licensing) may be performed individually or collaboratively.

Aspects of the invention may be described herein in the context of individual analytical steps, particular structural features, etc. However, it should be appreciated that any of the methods and devices described herein also may be incorporated into a business method associated with the use of a biomarker based on one or more blood vessel structural features or patterns.

Aspects of the invention relate to detecting and analyzing pattern parameters of in situ tubular structures (e.g., parameters of a subject's in situ vasculature). Aspects of the invention may be automated (e.g., using one or more computer-implemented acts described herein). It should be appreciated that one or more pattern parameters (e.g., individual blood vessel structural feature(s), distributions of blood vessels or blood vessel structural features, or combinations thereof) may be analyzed using one or more quantitative and/or qualitative methods. In some embodiments, one or more parameters may be measured and quantified and the measurements may be analyzed using standard quantitative and/or statistical techniques for evaluation and/or comparison with threshold or reference values as described herein. In certain embodiments, one or more parameters may be evaluated using a predetermined scoring method (e.g., based on predetermined factors). Geometrical parameters may be represented using vectors. For example, a distribution of blood vessels, blood vessel curvatures, blood vessel tortuosity, or blood vessel directions within a volume of interest may be represented using a plurality of vectors. Separate vectors may be used to represent separate vessels (e.g., vessels for which a connectivity has not been determined during the analysis). However, separate vectors also may be used to represent individual segments or fragments of a single blood vessel or portion of a vascular tree (e.g., for which connectivity has been or may be determined during the analysis). Vasculature pattern parameters may be analyzed using any appropriate technique for separating and/or categorizing numerical values or scores. Blood vessels of different sizes may be analyzed separately and compared to different threshold or reference values as described herein. For example, distributions of blood vessels or blood vessel structural features may be analyzed using a histogram or a curve representing a distribution of numerical values or scores of predetermined pattern parameters.

In one embodiment, a score may be obtained to relate a pattern parameter to the probability of a physiological condition such as a disease or a stage of a disease. Aspects of the invention can be used for in situ diagnostic, interventional and therapeutic analysis of one or more disease loci associated with aberrant internal structures. As used herein “in situ” means in an animal (e.g., a human) body as opposed to in a biopsy or other tissue sample. Aspects of the invention can be used to research structural changes associated with a disease, for developing and evaluating disease treatments including therapeutic drugs, and for other purposes. Aspects of the invention include automatically analyzing a structural feature or pattern and automatically generating a score based on the analysis.

In one embodiment, aspects of the invention include detecting and/or analyzing selected internal tubular networks in animals. As used herein, an internal tubular network means a network of connected cylindrical internal body structures. Tubular networks include, but are not limited to, cardio-vascular, respiratory, gastro-intestinal, and genito-urinary systems and portions thereof within animal bodies. Accordingly, the cylindrical structures may include branched, straight, curved, and/or twisted cylindrical elements. The cylindrical structures and elements may include not only cylinders, but also may include flattened or otherwise distorted regions. The cross-section of a cylindrical structure or element may be circular, oval, approximately circular, approximately oval, or more irregular in nature. The internal diameter of the cylindrical elements may vary or may be approximately the same over the region of interest. A tubular network such as a circulatory network may be closed off from the environment outside the animal. In contrast, tubular networks such as respiratory and gastro-intestinal networks may be open to the outside environment.

In one embodiment, aspects of the invention include analyzing a segmented tubular network (e.g., a segmented vascular network). In one embodiment, a segmented representation of a network, or a portion thereof, may be obtained (e.g., from an existing database or a remote site) and analyzed. In another embodiment, a segmented representation of a network, or a portion thereof, may be generated from structural data and then analyzed. According to aspects of the invention, an analysis may include detecting the presence or absence of one or more structural features or patterns, measuring or evaluating the extent of one or more structural features or patterns, or a combination thereof.

In one embodiment, aspects of the invention are useful for selectively detecting and/or analyzing patterns (e.g., structures) of an animal's vasculature to detect or monitor one or more blood vessel patterns (e.g., structures) that may be indicative of a physiological condition of the animal. A structural pattern or feature may be detected and/or analyzed for blood vessels of any size including, but not limited to, arteries, arterioles, veins, venules, and capillaries.

In one embodiment, aspects of the invention are useful for selectively detecting and/or analyzing structural features or patterns of an animal's vasculature to detect or monitor one or more blood vessel structures that are characteristic of disease (e.g., a disease associated with angiogenesis). A blood vessel structure or pattern characteristic of a disease (e.g., a disease associated with angiogenesis) may provide an early diagnostic indication of the presence of the, which can allow for early treatment that can improve a patient's prognosis. In other embodiments, a blood vessel structure or pattern characteristic of a disease (e.g., a disease associated with angiogenesis) can be used as a marker (e.g., a biomarker) for staging and/or grading, to monitor disease progression, evaluate a prescribed therapy, and/or identify and/or validate a drug or treatment regimen for the disease. Diseases associated with abnormal vasculature structures or patterns include, but are not limited to, cancer, cardiovascular, dermatologic (skin), arthritic, musculoskeletal, central nervous system, neurologic, pulmonary, renal, gastrointestinal, gynecologic, genitourinary, inflammatory, infectious, and immunologic diseases.

A cancer may be a solid tumor or a leukemia. When the cancer is a leukemia, methods of the invention may be directed to detecting and/or analyzing vasculature pattern(s) in the bone marrow of an animal (e.g., human).

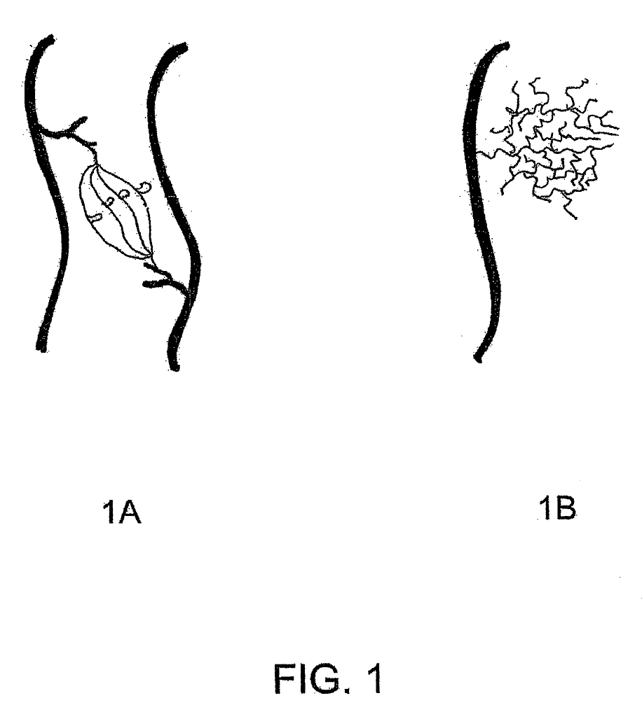

FIG. 1 illustrates a portion of an animal's vasculature as an example of a tubular network that can be analyzed according to some embodiments of the invention. FIG. 1A illustrates a healthy vascular network showing blood vessels of different sizes with a hierarchical pattern of branched blood vessels including capillaries. FIG. 1B illustrates a vascular network with structural signs of angiogenesis, including an area characterized by a dense and disorganized network of small blood vessels that are abnormally tortuous and branched. In one embodiment, aspects of the invention can be used to analyze selected in situ, in vivo, structural features or patterns of vascular networks such as those shown in FIG. 1. A score can be generated automatically to identify the presence of abnormal structure(s) and/or to evaluate the extent and/or degree of abnormality. In one embodiment, a map can be generated to represent the body region being analyzed and may include local scores that are useful to identify the location and extent of any abnormal vascular structure within the region. In one embodiment, aspects of the invention may involve an analysis (e.g., an automatic analysis) that detects abnormal structural features or patterns without representing and/or analyzing all of the vascular structures in a body region being analyzed. In one embodiment, information obtained regarding the location and/or extent of structural abnormality may be used to detect, identify, and/or evaluate a disease (e.g., including the type of disease, the extent of disease progression, and any specific stage of the disease, etc.)

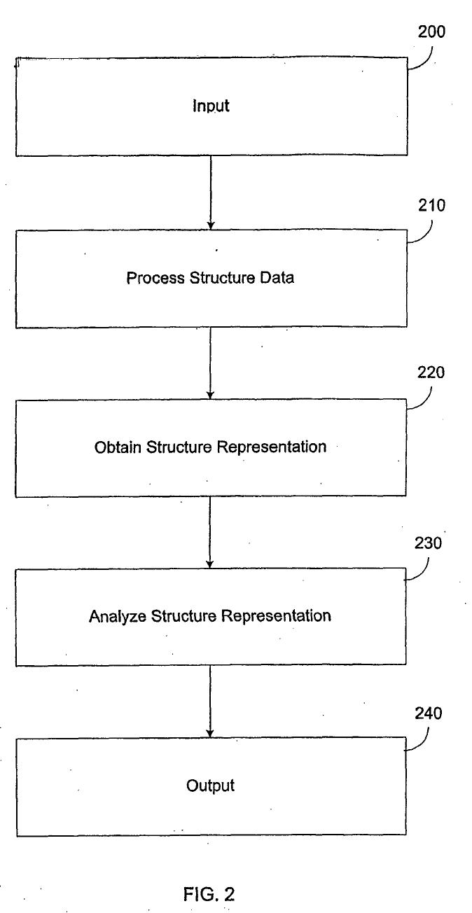

FIG. 2 illustrates a method for processing structure data in accordance with one embodiment of the invention. Initially, in act 200, input structure data relating to an internal animal structure is received. As used herein, structure data may be any form of data that can be used to identify and/or represent an internal animal structure. Accordingly, structure data may include the type of raw data obtained directly from an imaging device (e.g., scan data or view data), reconstructed image data (e.g., reconstructed from the scan or view data), model data (e.g., for a model that is configured to correspond to structures in scan or view data or in reconstructed image data), or a combination of any of the above. Examples of suitable imaging devices for providing the structure received in act 200 include non-invasive devices such as CT, rotational CT, micro-CT, multiple energy computed tomography (MECT), single detector CT (SDCT), multi-detector CT (MDCT), volumetric CT (VCT), MRI, micro-MR, X-ray, rotational X-ray, PET, near infrared/optical and other non-invasive scanning techniques and devices that may be used outside a subject's body or inserted non-invasively into a body cavity in order to detect internal structures in situ. Aspects of the invention described herein are not limited to use with structure data obtained in any specific way. Accordingly, structure data may be obtained by CT angiography (CTA), tomosynthesis, X-ray micro-angiography, or any other technique. Structure data may be obtained for an entire animal body, or may be obtained for one or more target volumes of the animal body. A target volume can be any portion of the animal body. In some embodiments, the target volume is an organ or a portion of an organ, e.g., a lung, liver, breast, colon, etc., or portion thereof. In other embodiments, the target volume can be a portion of the body, e.g., a limb, the abdomen, the torso, the neck, the head, or any portion thereof. A target volume can also include one or more bones in the animal body. According to aspects of the invention, a subject may be an animal and an animal may be any animal including a mammal, a bird, a fish, or a reptile. Mammals include, but are not limited to, humans, dogs, cats, rats, mice, goats, sheep, cows, horses, pigs, and monkeys. While applications in humans may be particularly valuable, experimental animals often may be used for research and development purposes. The animal may be a live animal, in which case the analysis relates to in situ, in vivo, structure. However, in one embodiment, aspects of the invention may be used to analyze structures (e.g., vascular structures) is dead animals (e.g., for a post-mortem analysis).

In one embodiment, structural data may be obtained and/or analyzed relating to cylindrical structures of interest (e.g., blood vessels) having an internal diameter of less than about 5 mm, preferably less than about 3 mm, more preferably less than about 1 mm, even more preferably less than about 500 microns, even more preferably less than about 200 microns, even more preferably less than about 100 microns, even more preferably less than about 50 microns, even more preferably less than about 20 microns, even more preferably less than about 10 microns, and even more preferably about 5 microns. However, tubular structures with smaller or larger internal diameters also can be analyzed according to aspects of the invention. In one embodiment, structure data obtained from one or more X-ray detector elements using conventional CT scanners may be processed to analyze tubular structures (e.g., blood vessels) with internal diameters ranging from above about 5 mm to below about 500 microns, and preferably below about 200 microns, and more preferably below about 100 microns. In another embodiment, data obtained from one or more flat-panel X-ray detectors using CT scanners such as micro-CT or VCT scanners may be processed to analyze tubular structures (e.g., blood vessels) with internal diameters ranging from about 100 microns to below about 50 microns, and preferably below about 20 microns, and more preferably below about 10 microns.

In one embodiment, structural data may be obtained from a human or other animal in a process that involves introducing at least one contrast agent into a body region of interest. For example, a contrast agent for detecting blood vessels may be injected into a blood vessel. A small amount of contrast agent may be introduced locally to enhance the detection of structures such as blood vessels in a particular body region of interest. Alternatively, a contrast agent may be provided in an amount sufficient to enhance the detection of structures such as blood vessels in a large body region or in the entire animal body. In other embodiments, structural data may be obtained without using a contrast agent.

In one embodiment, an analysis of structural data obtained from a suitable imaging device may involve generating a representation of one or more structures. The representation may be segmented so that only a subset of the structures in the structure data are represented (e.g., the representation may show only the vasculature and not the other structures around it). The segmentation may identify and/or detect the boundaries of defined structures (e.g., the boundaries of vasculature structures).

Act 210 is optional and may be used in some embodiments to perform any suitable processing operation on the structure data received in act 200. For example, structure data may have been obtained for the entire body of an animal, and the structure data may be processed at act 210 to select only data for one or more body volumes of interest. Alternatively, or in addition, the structure data may be processed so that it is in a more suitable format for processing in act 220. In accordance with one embodiment of the present invention, acts 220-240 (described in more detail below) that relate to analyzing information obtained from the structure data can be performed on one or more computing devices that are disposed remotely from the imaging device that obtains the initial structure data. In such an embodiment, the processing of the structure data in act 210 can include any suitable processing technique(s) that may facilitate transmission of the data to the remote processing device, including any suitable formatting for packaging changes, or any security techniques (e.g., encryption) to protect the data during transmission. However, it should be appreciated that any process described herein that may involve remote processing also may be performed locally without sending or receiving data, or any other form of information, to or from a remote site.

In act 220, a representation of an internal body structure (e.g., a vessel network or portion thereof) is obtained either from the initial structure data obtained directly from the imaging device or from segmented and/or processed structure data. The representation can be generated using any technique capable of producing a representation with sufficient resolution to glean the desired information therefrom in act 230 as discussed below. In one embodiment, the representation may be a representation that represents only one or more selected internal structures and does not represent all of the internal structures for which initial structure data from an imaging device was available. The segmented representation may be generated using only processed structure data described above, or the structure data may be processed as part of the technique used for obtaining a segmented representation.

In one embodiment, the structural representation is obtained by using techniques that enable the gleaning of information relating to one or more tubular structures having an internal diameter of less than about 5 mm, preferably less than about 3 mm, more preferably less than about 1 mm, even more preferably less than about 500 microns, even more preferably less than about 200 microns, even more preferably less than about 100 microns, even more preferably less than about 50 microns, even more preferably less than about 20 microns, even more preferably less than about 10 microns, and even more preferably less than about 5 microns. However, representations of tubular structures with smaller or larger internal diameters also can be obtained as the invention is not limited in this respect. In one embodiment, the structural representation is obtained using techniques that enables data obtained from one or more X-ray detector elements in conventional CT scanners to be used to glean information relating to one or more tubular structures (e.g., blood vessels) having internal diameters ranging from above about 5 mm to below about 500 microns, and preferably below about 200 microns, and more preferably below about 100 microns. In another embodiment, the structural representation is obtained using techniques that enable data obtained from one or more flat-panel X-ray detectors in CT scanners such as micro-CT or VCT scanners to be used to glean information relating to one or more tubular structures (e.g., blood vessels) with internal diameters ranging from about 100 microns to below about 50 microns, and preferably below about 20 microns, and more preferably below about 10 microns.

As mentioned above, in one embodiment of the present invention, the structure for which a representation is obtained may form part of a vascular network, which may include branched regions, curved regions, and/or regions having other structural features of interest. However, the aspects of the present invention described herein are not limited to generating representations of vascular networks, and can be employed on any suitable structure of interest.

It should be appreciated that the reference to obtaining a representation of a structure in act 220 does not necessarily require the creation of a visual representation on a visual display, but can also include the generation of a data set that includes sufficient information to specify the nature of the structure represented, such that as used herein, the reference to obtaining or generating a representation refers to obtaining (e.g., by generating) information specifying the nature of the structure of interest (e.g., the representation can be a reconstructed image, a model, or any other suitable form of representation). One example of a suitable technique for obtaining a structure representation is described in detail below, but the invention is not limited to using this technique, as any suitable technique can be employed. It should be appreciated that the process of obtaining a representation may involve data segmentation to obtain a representation of only specific structures or structural features of interest.

In act 230, one or more patterns (e.g., individual structural features or distributions) of the representation obtained in act 220 may be analyzed. The aspects of the present invention described herein are not limited to analyzing any particular patterns or features, as the particular patterns or features analyzed may vary depending upon the application of the techniques described herein. For example, when a vascular network is analyzed for the purpose of determining whether abnormal (e.g., cancerous) tissue is present or growing (e.g., angiogenesis), such patterns or features can include any of the curvature, tortuosity (degree of curvature and frequency of curves), density, branching (e.g., in an non-hierarchical way) and size (e.g., diameter or length) of the vessels under examination, diameter distribution, and geometric orientation of blood vessels or fragments of the blood vessels within a field, the distribution of the orientation and any combination of the foregoing. For example larger degrees of curvature and higher frequencies of curving may be associated with higher tumorigenicity or worse prognosis.

Thus, in accordance with one embodiment of the present invention, the representation of the structure being analyzed can be mined to identify one or more patterns (e.g., individual structural features or distributions) of interest. In one embodiment, the analysis of a structural representation may involve data segmentation in order to focus on a subset of one or more structures or patterns (e.g., individual structural features or distributions) of interest, so that all of the structures that are included in the representation need not be analyzed.

In one embodiment, a score may be generated to indicate the probability that the structural pattern(s) or feature(s) of a tubular network is (are) associated with a certain condition of interest, e.g., a healthy condition or a diseased condition. The score can be a quantitative or a qualitative score, and may be based on a single structural feature, or on two or more structural features, or on a distribution of structures or structural features, or any combination thereof. Scores can be based solely on one or more pattern parameters (e.g., structural features and/or distributions) gleaned from the representation, or can also be based upon additional information concerning the subject, including, but not limited to, age, weight, gender, medical history, genetic risk factors, exposure to disease causing agents, combinations thereof, etc.

In one embodiment, a score can be generated by assigning or calculating value(s) for one or more pattern parameters (e.g., structural features and/or distributions) included in the representation, and comparing the value(s) to one or more reference values. The reference values may be characteristic of a healthy subject, or of a condition such as a disease. A reference value(s) can be obtained in any suitable manner, such as by taking an average value obtained from the analysis of a plurality of individuals. In one embodiment, the reference value(s) can be adjusted for one or more subject parameters including, but not limited to, age, weight, ethnic background, genetic factors, gender, etc. or a combination thereof. In some embodiments, the reference value may be a subject-specific reference value obtained from one or more prior analyses of the same subject.

In another embodiment, a score can be generated by directly comparing one or more structures of interest in the representation to one or more reference structures. This can be achieved by any suitable comparison method, including graphic overlays, statistical analyses, or other suitable techniques.

In act 240, an output of the analysis is generated. The output can be provided in any form, including using alpha numeric characters, one or more tables, one or more graphs, one or more figures, a written report incorporating one or more of the foregoing, etc. The output can be displayed on a display device, communicated as an audio message, printed on paper or other tangible medium, provided in computer readable form or provided in any other suitable form. With respect to content, the output provided at 240 can similarly take any suitable form. For example, the content of the output can include a score as discussed above which can be used in any desired way, such as by a physician to provide a diagnosis or prognosis to a patient, by a researcher to evaluate the effectiveness of a treatment or candidate drug, or in any other suitable way.

In an alternative embodiment of the invention, the output can include a diagnosis, prognosis or other conclusion automatically generated in the act 230 as a result of analyzing the representation of the structure. In another embodiment, the output can be a representation of the region being analyzed including individual information (e.g., individual scores) for different parts of the region.

In yet another embodiment, the output can be a visual display (e.g., on a screen or other tangible medium including, but not limited to, a computer readable medium or a printed display) of at least a portion of the structure being analyzed, so that a user can look at the visual display and make judgments concerning the structure included in the subject.

It should be appreciated that the representations described herein in the context of any aspect(s) of the invention may be two-dimensional, three-dimensional, four-dimensional (e.g., with time as a fourth dimension, for example when analyzing a beating heart, or other changes over time such as disease progression or regression over time, etc.), or other multi-dimensional representations. Accordingly, the output can provide a multi-dimensional (e.g., at least a two-dimensional or three-dimensional or four dimensional, etc.) map of an animal body or portion thereof, with information relating to one or more patterns (e.g., individual structural features or distributions) and/or diseases associated with the map coordinates. It should be appreciated that these various types of output contents are not exclusive, and any combinations of the foregoing can be provided together, and furthermore that this list is not exhaustive, as the output can include any suitable content.

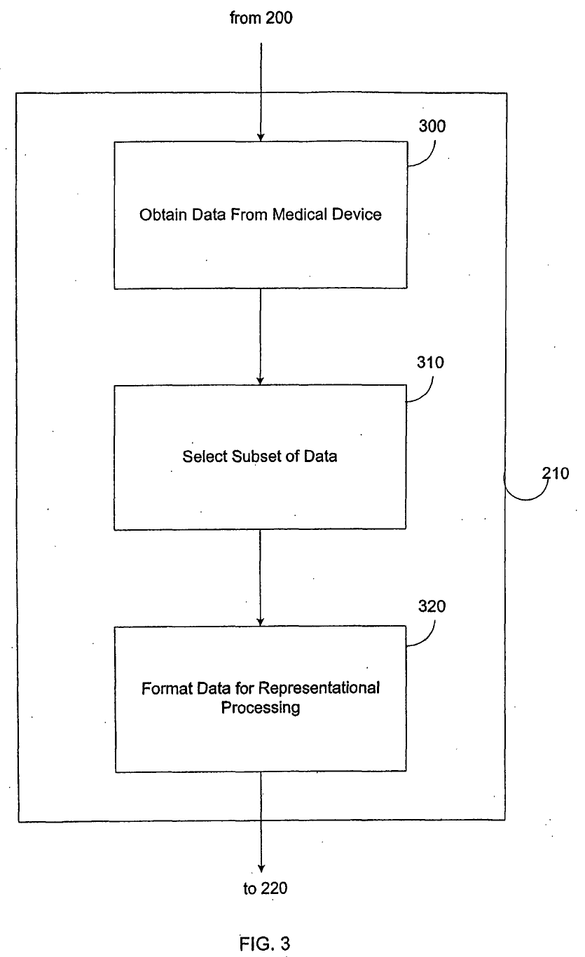

FIG. 3 illustrates one embodiment of a process for implementing act 210, where the structure data is obtained in act 300, a subset of the structure data is selected in act 310, and the data is formatted in act 320 into any suitable form for subsequent analysis or for secure transport or transmission as described herein. It should be appreciated that selection act 310 and formatting act 320 may be performed in any order. Also, both of these acts are optional, such that none, one, or both of acts 310 and 320 may be performed. In one embodiment, a subset of structure data may be selected in art 310 to focus on one or more specific regions, organs or tissue types of the body.

In another embodiment, the structure data may be processed to only select data relating to structures of a particular shape, range of shapes, size, or range of sizes. For example, the structure data may be processed to extract data relating to tubular blood vessels with an internal diameter below about 500 microns, preferably below about 200 microns, more preferably below about 100 microns, even more preferably below about 50 microns, and even more preferably below about 25 microns. It should be appreciated that the different data selection (including the regional/organ/tissue selection and/or size/shape selection) and formatting procedures described herein can be implemented together or separately in one or more processes (e.g., two or more sequential or parallel processes).

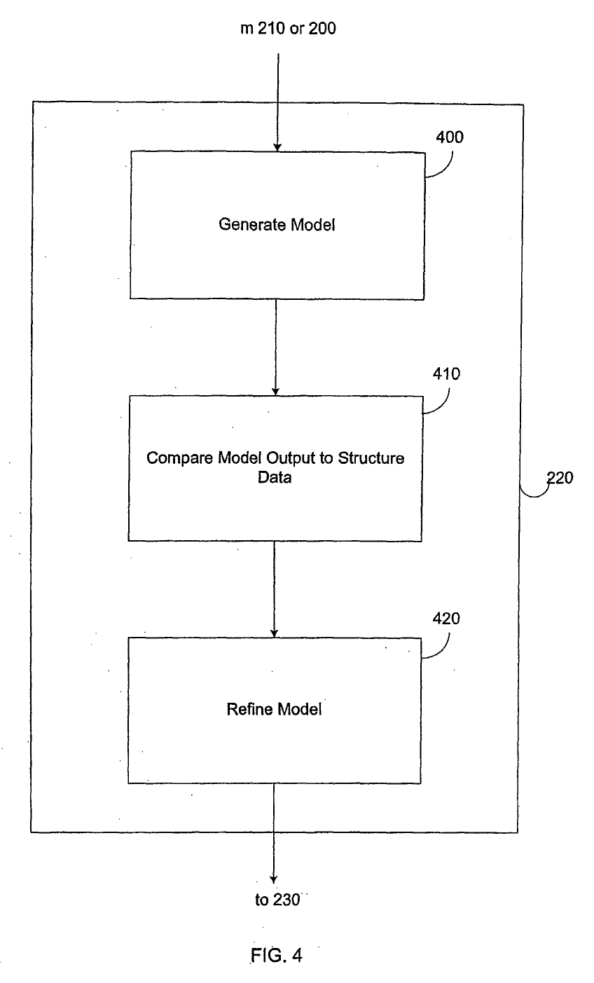

FIG. 4 shows one illustrative implementation for act 220 that generates a representation of at least one structure based upon initial or processed scan data. It should be appreciated that the particular implementation shown in FIG. 4 is described merely for illustrative purposes, and that the aspects of the present invention described herein are not limited in this respect, as a representation can be generated from structure data in any suitable way.

In the illustrative implementation shown in FIG. 4, model-based reconstruction is used to generate a representation from the scan data. Initially, in act 400, a model is generated that is believed to approximate one or more selected structures of interest for which information is contained in the scan data. For example, in one illustrative implementation described in detail below wherein the structures of interest relate to a vasculature network the model may comprise a plurality of cylinders that each approximates the shape of a vessel. In act 420, the model is compared to the scan data, and then in act 420 the model is refined based on the comparison. While not specifically shown in FIG. 4, the comparison 410 and refinement 410 acts can be performed in an iterative fashion to achieve a best fit between the model and the scan data using any suitable technique, examples of which are described below. Thereafter, the refined model can be presented for analysis in act 230, and provides a representation of one or more structure(s) included in the scan data. According to aspects of the invention, model-based reconstruction may automatically segment structure data by selectively processing only data that relates to one or more structures of interest.

It should be appreciated that aspects of the invention include performing individual acts described herein and do not require that more than one act be performed. For example, in one embodiment, an analysis may be performed on a structural representation that is present in a database or obtained from a remote location. Accordingly, aspects of the invention may involve analyzing a structural representation without generating the structural representation.

It also should be appreciated that aspects of the invention may include performing any combination of two or more acts described herein and that certain acts may be omitted in some embodiments. In one embodiment, the presence of one or more structural abnormalities may be identified or detected in a body region without generating and/or analyzing a structural representation of that body region. For example, the presence of a blood vessel abnormality may be detected directly from structure data for a body region without generating a structural representation of the vasculature for that entire body region. In another embodiment, an analysis may involve selectively representing one or more abnormal structures if they are present in a body region without representing normal structures in that body region (e.g., abnormal blood vessel structures may be represented without representing any normal blood vessels, or without representing all the normal blood vessels, without representing most of the normal blood vessels, etc.). In another embodiment, an abnormal vascular structure may be identified or detected without obtaining a detailed representation of the all the blood vessels in a body region. It may be sufficient to detect the presence of or outline of a vascular tree in a body region and perform an analysis that identifies or detects abnormal structures on specific blood vessels or the presence of excessive vascularization (e.g., a clump of neovasculature representing malignancy) without representing all the normal details of the vascular tree or even detecting individual blood vessels in the vascular tree. Accordingly, in some aspects a low resolution data set for a body region may be sufficient to detect or identify certain structural indicia of a disease such as cancer.

Aspects of the invention may include automating one or more acts. For example, an analysis may be automated in order to generate an output automatically. Acts of the invention may be automate using, for example, a computer system.

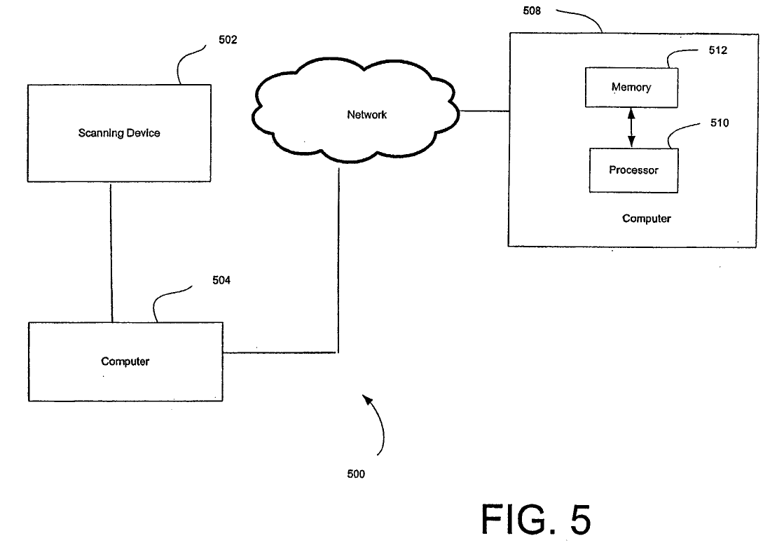

FIG. 5 illustrates a computer system on which aspects of the invention can be implemented. The computer system 500 of FIG. 5 includes a scanning device 502 that scans the subject of interest and generates scan data based thereupon. As discussed above, the scanning device 502 can take any suitable form, as the aspects of the present invention described herein are not limited to use with scan data provided using any particular type of scanning device.

In the illustrative system shown, the scanning device 502 is coupled to a computer 504, so that scan data can be transferred from the scanning device 502 to the computer 504 for any of the types of processing (described above, but the invention is not limited to use with such a system). For example, the processing performed by the computer 504 can be of the optional pre-processing described herein in connection with act 210 in FIG. 2. As discussed above, such pre-processing need not be performed, so that the scanning device 502 need not be connected to another computer for performing such a function. In addition, it should be appreciated that processing capabilities can be provided by the scanning device 502 itself, which may include a processor that can be programmed to perform any of the processing functions discussed herein.

In the illustrative system shown in FIG. 5, the computer 504 is coupled, via a network 506, to another computer 508, which includes a memory 510 and processor 512 for performing analysis on the structure data in any of the various ways discussed herein. The computers 504 and 508 can take any form, as the aspects of the present invention are not limited to being implemented on any particular computer platform. Similarly, the network 506 can take any form, including a private network or a public network (e.g., the Internet). A display device can be associated with one or more of device 502, and computers 504 and 508. Alternatively, or in addition, a display device may be located at a remote site and connected for displaying the output of an analysis in accordance with the invention. Connections between the different components of the system may be via wire, wireless transmission, satellite transmission, any other suitable transmission, or any combination of two or more of the above.

In accordance with one embodiment of the present invention for use on a computer system such as that shown in FIG. 5, it is contemplated that scan data can be obtained by a scanning device 502 and then sent over a public network, such as the Internet, to a remote location to be processed by computer 502 to produce any of the various types of outputs discussed herein (e.g., in connection with act 240 in FIG. 2). However, it should be appreciated that the aspects of the present invention described herein are not limited in that respect, and that numerous other configurations are possible. For example, all of the analysis and processing described herein can alternatively be implemented on the computer 504 that is attached locally to the scanning device 502, or on the scanning device 502 itself. As a further alternative, as opposed to transmitting structure data from the scanning device 502 to another computer 504 or 508 over a communication medium (e.g., the network 506), structure data (e.g., initial or processed scan data) can be loaded (e.g., via the scanning device 502 or computer 504) onto a computer readable medium that can then be physically transported to another computer (such as computer 508) for processing in the manners described herein. In another embodiment, a combination of two or more transmission/delivery techniques may be used.

As should be appreciated from the foregoing, in one embodiment, raw or processed structure data may be obtained at a medical or research center and sent to a computer at a remote site where one or more of the analytical steps described above may be performed (e.g., for a fee). The output from the analysis may be then returned to the medical or research center either in computer readable form to a computer at the medical or research center, in a hard copy, in another tangible form, or in any other suitable form including those described herein.

In another embodiment, one or more software programs that implement one or more functionalities described herein may be provided and installed at a medical or research center (e.g., for a fee). The programs can be provided on disk, downloaded from an internal or remote (e.g., external) site, or loaded in any suitable manner. Reference information that is used in any functionality described herein may be provided along with the software or separately. In one embodiment, reference information (e.g., information relating to normal or abnormal blood vessel structures) may be available on disk, downloaded from an internal or remote (e.g., external) site, or loaded in any suitable manner.

As used herein, “remote” means at a site that is different from the immediate location of the imaging device (e.g., the medical scanner). The remote site can be a central computer or computing facility at a hospital, medical, or research center (e.g., within the network or intranet of the center), or can be outside the hospital, medical, or research center (e.g., outside the network or intranet of the center). The remote site can be in the same state, in a different state, or in a different country from the site of data acquisition by the imaging device.

In some embodiments, multimodal analyses (e.g., using structure data from two or more different types of imaging devices) may be used together. Accordingly, aspects of the present invention may include the ability to process and analyze different types of structure data and either combine the results to generate a combined output, or to generate a separate output is generated for each imaging modality.

As discussed above, aspects of the invention described herein can be used for diagnostic, interventional, therapeutic, research, and treatment development and evaluation. Examples are described below.

Diagnostic Applications

In one embodiment, aspects of the invention can be used to detect and diagnose diseases associated with patterns (e.g., individual structural features or distributions) of in situ tubular networks. In some cases, a diagnosis can be rendered from an examination of the patterns (e.g., individual structural features or distributions) of interest at a single time. Alternatively, disease progression in a subject can be tracked by performing a structural analysis at two or more time points. Disease tracking can be used to provide diagnostic and prognostic information for a patient. For example, disease progression information can be used to assess the aggressiveness and/or invasiveness of a tumor.

The invention can be used to screen an individual or a population for the presence of indicia relating to one or more diseases. As mentioned above, the screen may be a whole body screen, or may be focused on one or more target regions (e.g., specific organs or tissues).

In one embodiment, the techniques described herein can be used automatically to identify individuals with one or more disease-associated structural patterns or features. These individuals can be subsequently tested for additional indicia of disease. The subsequent testing can take any suitable form, as the aspects of the present invention described herein are not limited in this respect. For example, follow on testing can employ conventional techniques. As a non-limiting example, the use of aspects of the present invention may enable cost-effective screening techniques that may identify a relatively small pool of candidates as at risk of a disease, and may justify the use of relatively more expensive testing procedures to reach a final diagnosis or prognosis, wherein the follow on techniques may be too expensive to administer to a wider sample that has not been narrowed using the techniques of the present invention described herein. As a further example, aspects of the present invention described herein, either alone or in combination with other techniques, can be used to perform subsequent tests. In this respect, the sensitivity of the initial screening can be set relatively high, such that it may indicate some false positives, and subsequent application of techniques in accordance with aspects of the present invention described herein can be employed with a higher degree of sensitivity that may provide more detailed information.

In one embodiment, aspects of the present invention can be used to screen a population of at risk individuals (e.g., individuals with genetic or other risk factors for a disease such as cancer, a circulatory disorder, or other disease) to identify the presence of disease indicia in one or more individuals.

In one embodiment, diagnostic methods of the invention are computer-implemented to increase efficiency and throughput, and reduce variability associated with individual physicians. However, as discussed herein, in some embodiments, the final diagnosis may be made by a physician based on information generated by an automated analysis or a structural representation using aspects of the invention described herein.

As shall be appreciated from the foregoing, aspects of the invention can be used on patients known to have a disease, or can be used to screen healthy subjects on a regular basis. A subject can be screened for one or more diseases. Screening can be done on a regular basis (e.g., weekly, monthly, annually, or other time interval); or as a one time event. Different conditions can be screened for at different time intervals and in function of different risk factors (e.g., age, weight, gender, history of smoking, family history, genetic risks, exposure to toxins and/or carcinogens etc., or a combination thereof).

In one embodiment, aspects of the invention can be employed to diagnose, evaluate or stage diseases associated with changes in vasculature structure. The detection of small changes in vasculature structure may be informative for early stage disease detection and disease monitoring. A high-resolution three-dimensional image of a vasculature structure may be analyzed and one or more patterns (e.g., individual structural features or distributions) may be evaluated for the presence of abnormal properties. In one embodiment, a vasculature structure may be a vascular tree including a series of interconnected branched blood vessels and may include arteries, arterioles, veins, venules, capillaries, and other sized blood vessels. According to aspects of the invention, different sizes of blood vessels can be detected and represented. In some aspects of the invention, the vascular tree of the entire body can be analyzed, and in other aspects the vascular tree of a target organ, tissue, or part thereof can be analyzed. In some aspects of the invention, a vascular tree containing only a subset of blood vessel sizes is analyzed (e.g., blood vessels with a diameter below about 500 microns, preferably below about 200 microns, more preferably below 100 microns, even more preferably below 50 microns, and even more preferably below 25 microns). In one embodiment, only capillary blood vessels are analyzed. In another embodiment, capillaries and small arteries and veins (e.g., arterioles and venules) are analyzed. For example, an arborescent vasculature can be analyzed in any tissue where it is found (e.g., an arborescent mucosal vasculature such as the oesophageal arborescent mucosal vasculature).

The branches of a vascular tree may be analyzed to glean information about the status of the patient. In one embodiment, the branches of a vascular tree may be followed to identify specific regions where certain characteristics of angiogenesis may be evaluated (e.g., start with a large branch and follow the tree to second, third, or fourth, or subsequent levels of branching to identify small blood vessels that may have abnormal structures if they are providing a blood supply associated with a disease). Alternatively, several different blood vessel sizes in the vascular tree may be evaluated for signs of angiogenesis. In another embodiment, the overall branching pattern of a vascular tree can be analyzed. For example, a healthy vascular tree may be approximately hierarchical in that the size of the blood vessels generally decreases as the vessels branch (e.g., FIG. 1A). In contrast, a diseased (e.g., angiogenic) vascular tree may be less hierarchical with areas of significant blood vessel branching with little or no decrease in blood vessel size (e.g., FIG. 1B). It should be appreciated that the nature and extent of the analysis may depend on the goal of the diagnostic evaluation. For example, a full body scan can be evaluated selecting all vascular structures and analyzing the entire vascular network for signs of different diseases. Alternatively, a region of a body suspected of being diseased may be selected and the data may be processed to focus on the vasculature in that region (e.g., to obtain a segmented representation of structures in the region of interest). A region of interest may be an organ (e.g., pancreas, liver, breast, colon etc.) or a tissue (e.g., skin epidermal tissue). The presence of an abnormal vasculature structure can be an early indication of a range of diseases for which early detection is critical for effective treatment.

Diseases associated with changes in vascular structure (e.g., that can be detected by the presence of abnormal vascular patterns at a given time or abnormal structural changes observed as a function of time) include, but are not limited to, cancer, heart diseases and related circulatory disorders, eye diseases, skin disorders, and surgical conditions. For example, diseases and conditions associated with changes in vascular structure include, but are not limited to, tumor angiogenesis, recurrent and progressive cancers, coronary artery disease, cardiomyopathy, myocardial ischemia, arteriosclerosis, atherosclerosis, atherosclerotic plaque neovascularization, arterial occlusive disease, ischemia, ischemic or post-myocardial ischemia revascularization, peripheral vascular disease (including diabetic retinopathy), thromboembolic diseases (e.g., stroke, pulmonary embolism, brain aneurisms, and deep venous thrombosis), claudication, rheumatologic disorders (e.g., arthritis), immune disorders (e.g., rheumatoid arthritis, vasculitis, Wegner's granulomatosis, and systemic lupus erythematosis (SLE)), pulmonary disorders (including, emphysema, COPD, idiopathic pulmonary fibrosis, pulmonary arterial hypertension, and other respiratory disorders), myeloma, vascular proliferative disorders, gastrointestinal disorders (e.g., Crohn's disease, ulcerative colitis, and inflammatory bowel disease (IBD)), gynecologic disorders (endometrial polyp, vaginal bleeding, endometriosis, dysfunctional uterine bleeding, ovarian hyperstimulation syndrome, preeclempsia, polycystic ovarian syndrome (PCO), cervical cancer, and cervical dysplasia), skin disorders (infantile hemangioma, verruca vulgaris, psoriasis, neurofibromatosis, epidermolysis bullosa, Stevens-Johnson syndrome, and toxic epidermal necrolysis (TEN)), eye disorders (macular degeneration, maculopathies, diabetic retinopathy, and retinopathy of prematurity (retrolental fibroplasia)) wound healing, inflammation associated with immune responses, ischemia including limb ischemia and cardiac ischemia, Alzheimer's disease and other disorders such as wound dehiscence, Buerger Disease (thromboangitis obliterans, arteriosclerosis obliterans (ASO), ischemic ulcers) multiple sclerosis, idiopathic pulmonary fibrosis, HIV infections, plantar fasciosis, plantar fasciitis, Von Hippel-Lindau Disease, CNS hemangioblastoma, retinal hemangioblastoma, thyroiditis, benign prostatic hypertrophy, glomerulonephritis, ectopic bone formation, and keloids.

These different diseases are characterized by different changes in vasculature structure. Accordingly, in one aspect of the invention, parameters and scoring methodologies are used to detect, diagnose, and monitor particular diseases and their related therapies based upon particular characteristics of vasculature structure indicative of the disease. Even within each disease category, different diseases can be characterized by different changes in vasculature structure. Accordingly, structure mining and scoring can be fine-tuned to increase the sensitivity for particular types of disease within a category (e.g., lung cancer score, breast cancer score, etc., can be developed). Patient-specific scoring parameters can also be developed to follow the progression of a specific disease or disorder in a patient.

Structural vasculature changes include changes in vascular architecture and vascular morphology affecting blood vessels and/or lymph vessels. Structural changes can involve neovascularization (including the growth of large blood vessels (e.g., arteriogenesis) and the growth of microvasculature (angiogenesis)), large blood vessel expansion, and vascular necrosis. Angiogenesis involves the formation of new blood vessels that sprout from preexisting blood vessels. Angiogenesis is different from vasculogenesis, which is the de novo formation of vessels that occurs primarily during development. Vasculogenesis is rarely associated with a disease or disorder. However, aspects of the invention can be used to study the natural process of vasculogenesis to help identify and understand defects in de novo blood vessel formation.

Angiogenesis is often associated with tumor growth and is a useful biomarker for cancer. Angiogenesis also can be associated with conditions where new blood vessel growth occurs in response to a reduced oxygen supply or blood flow (whether due to thrombosis, embolism, atherosclerosis, or other chronic occlusion or narrowing of the vasculature). Certain respiratory, cardiovascular, and inflammatory disorders also are associated with angiogenesis.

Angiogenic blood vessels have structural characteristics that are different from those of established blood vessels. For example, the branching patterns and tortuosity of angiogenic blood vessels are very different from those of normal blood vessels. These and other structural features are found predominantly in microvasculature and can be used for mining and scoring vasculature structural images. However, changes in larger blood vessels such as arteries and veins also may be associated with certain diseases or disease stages (e.g., growth and development of large tumors or late-stage tumors).

The vasculature that supports a tumor is typically associated with the connective tissue of the tumor (the stroma) that supports the malignant cells (in the parenchyma). A discussed above, tumor blood vessels are irregularly spaced and characterized by heterogeneous structural patterns or features. However, the formation of tumor blood vessels and other forms of angiogenesis may involve a series of characteristic stages (see, for example, Dvorak, 2003, American Journal of Pathology, Vol. 162:6, pp. 1747-1757, the disclosure of which is incorporated herein by reference in its entirety). Early stage angiogenesis may be characterized by vascular hyper-permeability, fibrin deposition and gel formation, and edema. This may result in the enlargement of micro-vessels such as venules. The cross-sectional area of an enlarged micro-vessel may be about 4 fold that of a normal micro-vessel. The perimeter of an enlarged micro-vessel may be about 2 fold that of a normal micro-vessel. Enlarged micro-vessels may occupy about 4-7 fold the volume of normal micro-vessels in a region of active angiogenesis. The appearance of enlarged micro-vessels may be followed by the appearance of “mother” vessels that are enlarged, thin-walled, serpentine, and hyper-permeable. Mother vessels may undergo a process of bridging whereby trans-luminal bridges are formed dividing the blood flow within the vessel into smaller channels. A developing mother vessel also may contain one or more glomerular bodies that may expand to divide the lumen of the mother vessel into several smaller channels that are typically tortuous. Bridging and glomerular body formation in mother vessels may lead to the appearance of small capillaries characteristic of angiogenesis. However, certain mother vessels persist as abnormally enlarged vessels with thin walls. These vascular malformations are often characterized by the presence of an asymmetric muscular coat and perivascular fibrosis. Small arteries and arterioles also may increase in size in diseased tissue. Aspects of the invention include detecting and/or monitoring any one or more of the blood vessel structural changes described herein. In one embodiment, the presence of one or more patterns (e.g., individual structural features or distributions) characteristic of new blood vessel formation may be used to detect or monitor a disease. In another embodiment, the presence of one or more specific patterns (e.g., individual structural features or distributions) may be used to determine the stage of angiogenesis (e.g., early-stage, mid-stage, late-stage, etc.) in a body region.

Accordingly, abnormal changes in blood vessel size (diameter and/or length) can be early signs of diseases such as cancer or other disease associated with an increased blood supply. Changes in blood vessel size may occur before any structural signs of angiogenesis appear. In one embodiment, aspects of the invention are useful to detect blood vessels (e.g., capillaries) that are swollen and/or longer than normal. For example, aspects of the invention are useful to detect abnormally long intrapapillary capillary loops in situ (e.g., associated with early stages of cancer in oesophageal mucosa).

In some embodiments, blood vessel changes indicative of necrosis in tumor tissues may be indicative of the aggressiveness of the tumor tissue and/or the likelihood of metastasis, and/or the responsiveness to therapy, and/or the efficacy of a therapeutic treatment (e.g., a candidate drug), and/or an therapeutic treatment selection and/or modification (e.g., a change in drug or dose for an individual patient). Accordingly, in situ patterns (e.g., individual structural features or distributions) indicative of necrosis may be useful biomarkers for patient prognosis. In certain embodiments, necrosis within a region of a tumor may be indicated by one or more of the following patterns (e.g., individual structural features or distributions) within that region: a collapse in blood vessel structure, poor vascularization (e.g., a low blood vessel density relative to other regions of the tumor or relative to the perimeter of the tumor), a change in blood vessel size or shape over time, a lower than threshold number of blood vessels, blood vessels (e.g., in the microvasculature or the capillaries) that are separated by a greater than threshold distance (e.g., by more than 100 microns, more than 150 microns, or more than 200 microns) within a volume of the tumor, micro-vessel diameter and/or density indicative of undervascularization, etc., or any combination thereof. In some embodiments, a volume of avascularization or undervascularization may be evaluated or quantified and used as an indicator of necrosis. It should be appreciated that other indicia of necrosis may be used, alone or in combination with blood vessel features. Other indicia may include indicia of tissue collapse or cavitation that may be visualized (e.g., using CT etc.) and/or indicia of tissue viability using one or more markers of metabolic activity (e.g., ones that may be analyzed using a PET scan, etc.).

Aspects of the invention may be used for the detection (e.g., the automatic detection) of necrotic areas in a subject (e.g., in a tumor in a subject). A necrotic region is an avascular region within the boundary of a diseased tissue. Methods of the invention may be used to detect (e.g., automatically) the transition between the vascularized diseased tissue and avascular region that defines the boundary of the necrotic region.

Aspects of the invention also may be used to detect or evaluate (e.g., automatically) a response to therapy. For example, a response to therapy (e.g., to a specific drug and/or a specific dosage of a drug, and/or to a combination of drugs and specific dosages of these drugs, etc.) can be detected and assessed as follows. Changes in the vascular patterns (e.g. vessel normalization/straightening, disappearance of smaller diameter vessels leading to lower micro-vessel density and to skewing of the vessel diameter distribution towards the larger vessels) may be detected and/or evaluated within the volume defined by the boundary of the diseased tissue and the boundary of the necrotic area. An increase in the absolute volume size of the necrotic area and/or the rate of such change while the total volume of the disease (e.g. tumor) volume stays constant may be detected and/or evaluated as an indicator that the therapy is effective. An increase in the ratio between the absolute volume size of the necrotic area and the total disease (e.g., tumor) volume and/or the rate of change in this ratio may be detected and/or evaluated and used as an indicator that the therapy is effective. A ratio of the diseased tissue volume and the necrotic region volume may be detected and/or evaluated and when it approaches 1 and the overall diseased tissue volume starts shrinking it provides an indication that a therapy is effective.

Structural representations of blood vessels can be mined to identify and evaluate certain patterns (e.g., individual structural features or distributions) that can be used to provide a score that is related to the probability that the blood vessels are normal or abnormal (e.g., disease associated). Patterns (e.g., individual structural features or distributions) for scoring blood vessels include, but are not limited to, the following: diameter, curvature, tortuosity (including, for example, the degree of tortuosity, the length of the blood vessel along which abnormal tortuosity is observed, etc.), variability or heterogeneity (including spatial variability or heterogeneity over distance or in a volume), branching shape or pattern, branching density, branching hierarchy, blood vessel density, distribution of vessel size (ratio of microvasculature to macrovasculature) a field effect (the presence of blood vessels bending towards a specific region), blood vessel diameter distribution, variability of the geometric orientation of blood vessels or fragments thereof, and the distribution of the orientation(s) within a field. The score may have more significance if two or more of these parameters are evaluated. In some embodiments, a score is generated using one or more of these structural parameters combined with additional information such as patient-specific medical information (e.g., age, weight, height, gender, etc.) and the presence of one or more additional indicators of disease such as a visible lesion on an X-ray or other image. In some embodiments, a score can be provided for a tumor. An example of a useful score is one that reflects the vascularity of a tumor. An abnormally high vascularity (measured as a higher than normal blood vessel number, density, length, or combination of the above) is generally indicative of a more aggressive or invasive tumor. In one embodiment, vascularity is evaluated by measuring the volume of the lumen of angiogenic vasculature (the volume within the blood vessel tree associated with a tumor). In another embodiment, a measure of vascularity is provided by dividing the volume of the angiogenic lumen by the volume of the solid tumor. Additional information can be gleaned from obtaining a score (or other structural evaluation) at two or more times. A changing score (or other structural evaluation) is indicative of an evolving vasculature that could be associated with a disease or disorder. It should be appreciated that the patterns (e.g., individual structural features or distributions) described herein can be identified and analyzed for a field of analysis without imposing a connectivity on the vessels being studied. In some embodiments, it may be sufficient to analyze only fragments of blood vessels in order to detect one or more structural features of individual vessels or geometrical features of a field of vessels that are different from normal features. For example, blood vessel fragments having an average length of 0.5 mm, 1 mm, 5 mm, 10 mm, 50 mm, 1 cm, 5 cm, 10 cm, 50 cm, etc. may be used. However, it should be appreciated that shorter or longer or intermediate lengths may be used.

The scoring and mining aspects of the invention described herein can be automated. Accordingly, diseased (e.g., angiogenic) vasculature can be automatically detected amidst normal vasculature. Various vasculature parameters can be automatically detected and scored, either separately or in any combination, including vessel tortuosity, vessel branching, vessel density, and total intra-vascular volume, but the invention is not limited to any particular parameter or combination.

In one embodiment, aspects of the invention can be used to detect blocked blood vessels, and thromboembolic events, including stroke, lung emboli, blocked micro-coronaries, deep-vein thrombosis, etc. Blocked blood vessels can be detected (I) directly by detecting structural changes in the blocked blood vessel (e.g., detecting a clot, wall thickening, or other signs of reduced flow) and/or (2) indirectly by detecting new vasculature that was generated in response to the blockage. In general, the formation of collateral blood vessels is more ordered than angiogenesis associated with cancer. One aspect of the invention described herein also allows clots to be detected in small blood vessels.

As discussed above, aspects of the invention can be used to screen the entire vasculature structure of a human or other animal to screen for any form of abnormality in any tissue. Alternatively, a subset of the body may be screened. Accordingly, vasculature structures such as a vascular tree can be analyzed for one or more organs or tissue types. In addition, only a portion of the vasculature may be analyzed within any target volume as opposed to the entire vascular tree in that volume. This may be done by analyzing structure data focused on the area of interest, or large amounts of structure data may be obtained, but an analysis may be restricted to a subset of the available data. In some embodiments, only a portion of a vascular tree may be represented and/or analyzed, for example only those vessels that are of a particular size. In other embodiments, only fragments of a vascular tree are represented and/or analyzed if the fragments are sufficiently informative to provide patterns (e.g., individual structural features or distributions) of interest. Fragments may include branches or may be unbranched. The portion of the vasculature being analyzed may be statistically significant, such that any observation (normal or abnormal) is physiologically significant. For example, branched structures may not be required for the analysis if a sufficient number of vessel substructures are analyzed to confidently detect any other patterns (e.g., individual structural features or distributions) that may be associated with vasculature changes (e.g., angiogenesis) such as high vessel density. In aspects of the invention, vascular patterns may be detected and/or evaluated in situ in a volume of 1 mm³, 2 mm³, 5 mm³, 1 cm³, 2 cm³, 5 cm³, 10 cm³, etc. However, smaller or larger or intermediate volumes also may be analyzed.

Different tissues and organs have different and characteristic blood vessel patterns (e.g., the lung which is highly vascularized). Accordingly, in one embodiment, structural analyses and associated structural parameters may be optimized for evaluating different tissues.

In some embodiments, scan data is obtained and/or analyzed for one or more organs (e.g., lung, heart, colon, brain, liver, pancreas, kidney, breast, prostate, etc.) or tissue (e.g., skin, bone, etc.) or portion of any of the above.

Brains may be evaluated for signs of brain tumors and/or other neurological disorders that can be associated with changes in vascular patterns. For example, Alzheimer's may be associated with certain vascular abnormalities. In one embodiment, one or more changes in blood vessel pattern (e.g., shape and/or size) may be detected as an indicator of high blood pressure in the brain.

In some embodiments, certain specific regions of organs or tissues are focused on. For example, atherosclerosis is typically found in certain parts of the arterial tree (e.g., bifurcations, side branches, regions opposite flow dividers, and other areas where angiogenesis often occurs in association with atherosclerosis) and certain cancers tend to occur more frequently in certain organ or tissue regions (e.g., colon cancers are not distributed evenly along the length of the colon).

In other embodiments, aspects of the present invention may be used to follow up with individuals who have been identified as having one or more other indicia of disease (e.g., fecal occult blood, a colon polyp, a lung nodule, one or more cysts or other indicia of disease). Aspects of the invention may be used to confirm the presence of a disease, determine a location for the disease-associated lesion, or provide an evaluation or prognosis of a disease. For example, aspects of the invention may be used to determine whether abnormal vasculature is present at the site of a lesion (e.g. a colon polyp, a lung nodule, a bladder cyst, a prostate cyst, a breast cyst, a spot on a mammography, or any other cyst, lump, or spot that may be detected physically, visually, or using any other diagnostic technique) and help evaluate the likelihood of a malignancy (or other carcinogenic disease stage) associated with the lesion. Accordingly, aspects of the invention may be used for virtual malignancy detection (e.g., virtual colonoscopy, virtual colon malignancy detection, virtual bronchoscopy, virtual lung malignancy detection, virtual mammography, virtual cystoscopy, etc.).

In other embodiments, aspects of the invention may be used for screening a cancer patient to evaluate the extent of a cancerous lesion and/or to screen for the presence of one or more metastatic lesions (e.g., one or more loci associated with angiogenesis). A cancer patient may be screened upon initial diagnosis of a primary cancer. In addition or alternatively, a cancer patient may be screened at least once after an initial cancer treatment (e.g., surgery, radiation, and/or chemotherapy). This screening may include the original cancer locus to detect any cancer recurrence. This screening may include similar body tissue to screen for the presence of other lesions in the same tissue or organ (e.g., the entire colon may be screened when a cancerous lesion is detected in one region of the colon, the second breast may be screened when a cancerous lesion is detected in one breast, etc.). This screening also may be extended to the whole body or to one or more other loci suspected of containing a metastatic lesion. In one embodiment, a cancer patient may be screened several times after an initial cancer treatment (e.g., at time intervals of about 6 months, about 1 year, about 2 years, about 5 years, or at other time intervals).

In one embodiment, a follow up procedure may involve screening one or more organs or tissues for the presence of a metastatic lesion. Different cancers may have different characteristic patterns of metastasis. Accordingly, different target loci may be screened for different cancers. For example, metastatic breast cancer typically spreads to the lungs, the liver, bone, and/or the CNS. Therefore, one or more of these tissue types or organs may be screened after a patient is diagnosed with breast cancer. Similarly, other target loci may be screened after a patient is diagnosed with another cancer type. In some embodiments, the entire body of a cancer patient may be screened for indicia of metastasis.

In one aspect, an initial screen may be performed on an entire body, or an entire organ, using a low resolution representation and/or, for example, analyzing only one or two or a small number (e.g., less than five) pattern parameters in order to detect indicia of a disease. Subsequently, the presence and or nature of the disease may be diagnosed using a higher resolution representation and/or, for example, analyzing one or more additional pattern parameters or alternative pattern parameters than those that were analyzed for the initial detection.

It should be appreciated that some or all of the diagnostic aspects of the invention can be automated as described herein.

Interventional Applications

Aspects of the invention also can be used to identify the location of a disease by locating one or more structural abnormalities associated with the disease. This information can be used to target a biopsy procedure or a treatment (e.g., a treatment with one or more toxic chemicals, radiation, heat, cold, small molecules, gene therapy, surgery, any other treatment, or a combination of two or more of the above) to the precise location of a disease lesion, or for any other purpose.

In one embodiment, an imaging device is connected to a computer that provides a real-time visual display of the disease lesion. In one embodiment, a real-time visual display may be an accurate model of a body region and lesion along with associated vasculature (as opposed to an actual image). This visual information can be used to guide a surgical instrument for a biopsy. Alternatively, the information can be used to guide an invasive (e.g., surgical removal or bypass) or non-invasive (e.g., radiation) treatment procedure to the site of the disease lesion (e.g., tumor or blood clot).

In one embodiment, aspects of the invention may be used to identify an area of tissue for treatment before the treatment is applied. For example, a treatment target region may be identified by detecting a boundary of chaotic blood vessel structures. The area may be assessed after treatment to confirm that the treatment was appropriately targeted. In one embodiment, a structure may be analyzed pre-operatively to identify the extent of tissue to be removed from a body region. In one embodiment, a body region may be analyzed post-operatively to determine whether any abnormal structures were missed. This may be used to confirm the success of a radiation treatment or a surgical removal of diseased tissue. Alternatively, this may be used to decide on further surgery and/or another form of treatment. In another embodiment, a disease boundary may be defined or depicted by the boundary of abnormal vasculature. A treatment (e.g., radiation therapy, surgery, etc.) may be guided by and/or restricted to a volume encompassed by the disease boundary.

In one embodiment, aspects of the invention can be used to evaluate the success of a surgical implant or transplant. For example, aspects of the invention can be used to evaluate the formation of new blood vessels after an organ or tissue transplant.

In another embodiment, the development of new blood vessels may be monitored after removal of tumor tissue or after a tumor biopsy, both of which may trigger angiogenesis and/or convert a dormant tumor into a malignant tumor.

It should be appreciated that some or all of the interventional aspects of the invention can be automated as described herein.

Therapeutic

Aspects of the invention also can be used to optimize a therapeutic treatment for a patient. The extent of disease progression or regression can be monitored in response to different treatment types or dosages, and an optimal treatment can be identified. The optimal treatment may change as the disease progresses. The effectiveness of the treatment over time can be monitored by analyzing changes in disease-associated patterns (e.g., individual structural features or distributions) using the aspects of the present invention described herein.

In one embodiment, a first therapy can be administered and its effectiveness on slowing, stopping, or reversing abnormal blood vessel growth can be monitored either irregularly or at certain time intervals (e.g., daily, weekly, monthly, or other time intervals). In some embodiments, if a first therapeutic regimen does not have a desired effect on disease progression, a second therapeutic regimen can be evaluated. Similarly, additional therapeutic regimens can be evaluated on a patient-by-patient basis. Additionally, the invention can be used to optimize a chosen therapeutic regimen (e.g., optimize dosage, timing, delivery, or other characteristic of a drug or other treatment) by monitoring the effect of minor therapeutic changes and using the conditions that appear to be most effective for the condition and the patient.

When looking at the therapeutic effectiveness of a treatment, disease-specific parameters may be monitored. Of course, all parameters can be obtained and only a subset reviewed. However, it may be more efficient to simply obtain (a representation of) only those parameters that characterize the disease.

According to aspects of the invention, patterns (e.g., individual structural features or distributions) that are used to detect angiogenic vasculature and other abnormal blood vessels also can be used to monitor a disease response to treatment. For example, the total vascularity or any other volumetric analysis of angiogenic or other diseased vasculature, and the distribution of vessel size (e.g., a ratio of small to large blood vessels) can be used independently or together as indicators of disease progression or regression. In general, microvasculature disappears before macrovasculature if an anti-angiogenic treatment (or other disease treatment) is effective. Therefore, an effective treatment results in a shift in the distribution of blood vessel sizes towards larger vessels. An index of anti-angiogenic activity can be scored as either a loss of small blood vessels or a shift of observed blood vessels towards a single size (or both).

In another aspect, the parameters can be (or include) changes over time. For example, a structure present at a second time can be compared to a structure present at a first time. In one embodiment, a disease may be tracked pre-therapy and/or post-therapy. Naturally, additional time points can be used. The time points may depend on the condition being observed (e.g., is it the progression of a disease that is already identified, is it the screening of patient(s) over time). Time periods can be daily, weekly, monthly, annual, or shorter, intermediate or longer time periods. Time intervals may be a series of regular time periods. However, other time intervals may also be useful. In one embodiment, a patient-specific baseline is established and monitored over time. For example, vasculature changes in the colon, breast, or other tissue or organ can be monitored periodically.

In one aspect of the invention, a type of treatment may be determined by the degree or extent of abnormal vascular structures (e.g., angiogenesis) that is detected at one or more suspected disease loci (e.g., cancerous loci). For example, if a suspected cancerous locus or metastasis is pre-angiogenic or associated with early stage angiogenesis, it may be appropriate to monitor the locus without any form of treatment. However, an appropriate therapy may involve the administration of one or more angiogenesis inhibitors to prevent the formation of any new vasculature. If a suspected cancerous locus or metastasis is associated with mid-stage angiogenesis, an appropriate therapy may be the administration of one or more angiogenesis inhibitors. A patient with mid-stage angiogenesis at a suspected locus also should be monitored so that any further blood vessel development can be treated more aggressively. If a suspected cancerous locus or metastasis is associated with late stage angiogenesis, an appropriate treatment may involve at least one or more of chemotherapy (e.g., cytotoxic chemotherapy and/or hormone-based chemotherapy), radiation, surgery, and/or treatment with one or more angiogenesis inhibitors. However, it should be appreciated that any of the above treatment options may be used to treat a patient with any one or more lesions associated with any degree of angiogenesis.

Examples of angiogenesis inhibitors include but are not limited to 2-methoxyestradiol (2-ME), AG3340, Angiostatin, Angiozyme, Antithrombin III, VEGF inhibitors (e.g., Anti-VEGF antibody), Batimastat, bevacizumab (avastatin), BMS-275291, CAI, 2C3, HúMV833 Canstatin, Captopril, Cartilage Derived Inhibitor (CDI), CC-5013, Celecoxib (CELEBREX®), COL-3, Combretastatin, Combretastatin A4 Phosphate, Dalteparin (FRAGIN®), EMD 121974 (Cilengitide), Endostatin, Erlotinib (TARCEVA®), gefitinib (Iressa), Genistein, Halofuginone Hydrobromide (TEMPOSTATIN™), Id1, Id3, IM862, imatinib mesylate, IMC-IC11 Inducible protein 10, Interferon-alpha, Interleukin 12, Lavendustin A, LY317615 or AE-941 (NEOVASTAT™), Marimastat, Maspin, Medroxpregesterone Acetate, Meth-1, Meth-2, Neovastat, Osteopontin cleaved product, PEX, Pigment epithelium growth factor (PEGF), Platelet factor 4, Prolactin fragment, Proliferin-related protein (PRP), PTK787/ZK 222584, ZD6474, Recombinant human platelet factor 4 (rPF4), Restin, Squalamine, SU5416, SU6668, SU11248 Suramin, Taxol, Tecogalan, Thalidomide, Thrombospondin, TNP-470, TroponinI, Vasostatin, VEG1, VEGF-Trap, and ZD6474.

Some embodiments may include a method of selecting a subject for treatment and/or selecting a treatment or a course of therapy based on the analysis of certain in situ vascular structures. A method may involve analyzing in situ vascular structure(s) in a human subject to obtain, for example, a score. The score may be compared to a control score (e.g., in an apparently healthy population) or to a previous score from a previous analysis on the same subject. The treatment or the course of therapy may be based on such a comparison. In some embodiments, obtaining an analysis of vascular structures is repeated so as to monitor the human subject's response to therapy over time. In some embodiments of this aspect of the invention, the method further comprises measuring a second index of disease in the human subject wherein deciding on the treatment or course of therapy is also based upon the measurement of said second index.

In certain embodiments, patients having a tumor that is under-vascularized (e.g., one that shows signs of necrosis) may be selected for treatment with one or more anti-angiogenic compounds. Under-vascularized tumors may be identified as those that have a low density of blood vessels, or for which the blood vessel diameters are low (e.g., below a threshold number typical of vascularized tumors).

Aspects of the invention also may include monitoring the effectiveness of a therapy by monitoring the presence of blood vessel patterns or features over time. For example, the progressive loss of blood vessels in a tumor in response to treatment may be a sign that a therapy is effective. In contrast, the absence of any impact on vascularization may be an indicator that a treatment is not being effective in a patient and that an alternative therapy should be considered or used.

It should be appreciated that some or all of the therapeutic aspects of the invention can be automated as described herein.

Research

In one embodiment, aspects of the invention can be used to understand structural changes associated with biological processes of interest (e.g., disease development and progression). For example, an animal's vasculature can be analyzed to identify additional patterns (e.g., individual structural features or distributions) that may be associated with wound healing or different diseases or different disease stages. These additional patterns (e.g., individual structural features or distributions) may be used in one of more of the diagnostic, intervention, therapeutic, and development aspects of the invention.

In one embodiment, aspects of the invention can be used to understand structural changes associated with medical procedures. For example, an animal's vasculature can be analyzed to identify changes associated with post-surgical wound healing or implant/transplant (including xenografts) growth or rejection.

It should be appreciated that some or all of the research aspects of the invention can be automated as described herein.

Development and Evaluation of New Treatments Including Drug Screening and Validation

In another embodiment, aspects of the invention can be used in screens of compound libraries or to validate-candidate compounds for treating diseases associated with abnormal internal structures (e.g., abnormal tubular networks). Aspects of the invention allow efficient high throughput analyses of internal structural changes. These changes can act as surrogate markers (biomarkers) for certain diseases. As a result, the screening process can be automated to a large extent, and the time for obtaining results significantly shortened when compared to current validations that often involve waiting for disease symptoms to change and also may require tissue biopsies.

Surrogate markers: Aspects of the invention may be used for identifying and quantifying vascular patterns (e.g., structural features) that can be used as surrogate markers for diagnostic, therapeutic, and research and development purposes. Surrogate markers are useful for reducing the time of diagnosis, therapy evaluation, and drug development. A surrogate marker can be used as an early indicator for disease diagnosis, disease prognosis, or drug effectiveness, without waiting for a clinical outcome (e.g., increased survival time in response to a drug). So, a vasculature analysis can be used as a surrogate marker for drug development (in both pre-clinical and clinical trials), for clinical screening (e.g., breast, lung, or colon screening), and for clinical therapy monitoring. For example, vasculature structure is a useful surrogate marker for angiogenesis related diseases such as cancer.

In one embodiment, aspects of the invention provide methods for screening and/or validating candidate compounds or therapies for their effectiveness in treating neo-vasculature formation and/or vasculature pattern changes associated with disease. Aspects of the invention may be used to evaluate individual or small numbers of compounds or to screen libraries to evaluate and/or identify a plurality of candidate compounds (e.g., by administering these compounds, individually or in groups, to an experimental animal such as a mouse and evaluating their effect on angiogenic vasculature). Libraries may contain any number of compounds (e.g., from approximately 100 to approximately 1,000,000) Different types of compounds can be screened, including antibodies, small molecules etc. However, the invention is not limited by the number and/or type of compounds that can be evaluated.

In one embodiment, the effectiveness of a candidate compound can be compared to a reference compound. A reference compound can be any compound with a known effect on a structure. For example, Avastin (Genentech) is a known monoclonal antibody against vascular endothelial growth factor (VEGF) that can be used as a reference to test the effect of a candidate compound on neovasculature growth.

In vivo models: According to aspects of the invention, compounds and therapies can be evaluated in the context of an in-vivo model such as an animal disease model. For example, a mouse with cancer or atherosclerosis can be used to evaluate, optimize, and identify useful therapies. Other animal models also can be used. Aspects of the invention may be useful for high-throughput analyses because they can detect small changes in vasculature and can be used to evaluate a therapy in a short time period with minimal manipulation since little or no invasive procedures are required.

Vascular analysis aspects of the invention can be used on an orthotopic model to test, for example, the effectiveness of a drug in a short period of time. For example, the effect of a candidate drug on angiogenesis in an orthotopic mouse tumor model may be quantifiable after about 5 days (e.g., between 1 and 10 days, depending on the model and the drug). In contrast, a subcutaneous cancer animal model requires approximately one month for tumor growth to be analyzed and compared to controls.

An orthotopic model can be used to model different diseases or clinical conditions. Examples include, cancer, tissue regeneration, wound healing (including healing after traumatic injury, healing after surgical intervention, healing of burnt tissue such as skin), tissue or organ transplant therapy, medical device implant therapy, other conditions associated with neovascularization or changes in normal vascular structure, or any combination of two or more of the above. However, the invention is not limited by the type of orthotopic model or the type of disease or clinical condition that is being analyzed.

A single orthotopic disease model animal may be useful for testing more than one candidate drug molecule since the analysis does not involve sacrificing the model animal. Accordingly, once a test with a first candidate is complete, a subsequent candidate can be evaluated in the same model animal. A series of candidates can be tested in a single model animal, with appropriate controls, provided the model retains features of neovascularization that are necessary for the assay.

It should be appreciated that some or all of the development aspects of the invention can be automated as described herein.

It also should be appreciated that any one or more structural parameters described herein may be evaluated by comparison to a reference parameter. In some embodiments, a reference parameter may be an amount or score for that parameter in a normal or healthy subject. In other embodiments, a reference may represent a diseased condition. In some embodiments, a change or amount of any structural parameter that is correlated or associated with a disease or condition as described herein may be a statistically significant change or difference in that parameter in a diseased or test subject relative to a reference subject. In some embodiments, a difference or change in a structural parameter may be an increase or a decrease in a particular parameter (or a combination of parameters). An increase in a parameter may be at least a 5%, 10%, 20%, 30%, 40%, 50%, 60%, 70%, 80%, 90%, 100%, or greater increase in that parameter in a test subject relative to a reference subject. Similarly, a decrease in that parameter may be at least a 1%, 5%, 10%, 20%, 30%, 40%, 50%, 60%, 70%, 80%, 90%, 100%, or greater decrease of a measure of that parameter in a test subject relative to a reference subject. Once an amount of change or difference in a parameter has been correlated or associated with a disease or condition, that level may be used in subsequent methods according to the invention. Accordingly, in some embodiments, a difference of at least at least 1%, 5%, 10%, 20%, 30%, 40%, 50%, 60%, 70%, 80%, 90%, 100%, or more of any given structural parameter (e.g., tortuosity, density, volume, or any other individual structural feature or distribution of structures or structural features as described herein) relative to a reference value may be used as a threshold for methods of the invention. It should be appreciated that higher or lower or intermediate values may be used. It also should be appreciated that different parameters may have different threshold or reference levels. Also, different parameters (and/or different levels for each parameter) may be associated with different conditions or diseases. Accordingly, specific disease or condition values or thresholds may be identified for different parameters or combinations thereof. These threshold values may be used for disease detection, diagnosis, monitoring, or for any other therapeutic, clinical, or research application described herein (e.g., in automated methods described herein).

### EXAMPLES

### Example 1

The following example illustrates how aspects of the invention can be used for diagnostic, therapeutic, and research purposes by analyzing vascular structures associated with different diseases. However, it should be appreciated that the techniques described herein can be applied to different structures and for different diseases or conditions.

Bone analysis: Breast cancer often metastasizes to bone. However, there is currently no consensus on the optimal method for detecting a bone cancer lesion. In one embodiment, aspects of the invention can be used to diagnose a bone lesion and evaluate its response to treatment by analyzing blood vessel structures (and/or changes therein) in bones. A bone lesion can be of any type including osteolytic, osteoblastic, or a combination thereof. Lesions in the bone marrow can also be identified, diagnosed, and/or evaluated. Bone has a typical vasculature that is readily recognized. Using techniques described herein, changes in the vasculature and new vascular features can be distinguished from normal bone vasculature.

Certain conventional bone scan techniques such as PET use radio-labeled markers to identify cancerous tissue. However, such scans are complex and expensive, and are used only when there is a specific concern about the potential presence of a cancerous lesion in the bone of a patient. Aspects of the invention described herein do not require radio-labeled markers and provide structural information that may be easier to interpret and can be evaluated automatically. Bone vasculature analysis may be particularly useful for breast cancer patients to detect any early signs of cancer metastasis to bone loci. However, aspects of the invention may also be used to screen healthy subjects to detect any signs of vascular changes in their bones.

It should be appreciated that aspects of the invention also provide information that is useful for evaluating the stage of a bone cancer and for optimizing treatment for bone cancer.

Diabetic retinopathy: Diabetic retinopathy results from the formation of new blood vessels in patients with diabetes. Diabetic retinopathy causes retinal malfunction and visual complications leading progressively to blindness. If detected early, diabetic retinopathy can be treated or managed. For example, laser photocoagulation therapy can be used to prevent vision loss if blood vessel proliferation is detected early. In one embodiment, aspects of the invention can be used non-invasively to detect early blood vessel proliferation associated with diabetic retinopathy. The techniques described herein may enable the detection of earlier signs of neo-vascularization than methods such as fluorescein angiography or fundus photography. In addition, some embodiments of the invention do not require that a specialist be present at the same medical center as the patient, as detection and diagnosis may be performed at a remote location based on retinal blood vessel structural information derived from the patient.

Aspects of the invention also can be used to monitor and optimize therapeutic treatments to prevent or minimize vision loss in a diabetic patient. In particular, vascular structural information may be used to target a treatment to a region of the retina that is affected by early stages of diabetic retinopathy. The monitoring and treatment aspects also may be coordinated by a specialist at a remote location.

Lung Cancer: Lung cancer is a leading cause of cancer death, and early detection is the most effective technique for improving the chance of survival. Lung cancer shows up as pulmonary nodules on conventional two-dimensional chest radiographs and three-dimensional CT scans. However, aspects of the invention may be used to detect early changes in lung vasculature that appear before pulmonary nodules can be detected using conventional techniques.

In one embodiment, a subject's lung vasculature may be analyzed according to aspects of the invention to complement or confirm the diagnosis of a lung cancer that was initially detected using current chest X-ray or CT analytical techniques. The presence of abnormal vasculature at the same location as a spot on an X-ray may confirm the presence of a tumor at that site.

In another embodiment, aspects of the invention may be used as an initial screen to identify abnormal lung vasculature. It should be appreciated that if a pocket of angiogenic blood vessels is detected, follow up analyses may be performed using current chest X-ray or CT scan techniques. However, if the angiogenic blood vessels are detected early, cancer spots may not be visible using current non-invasive techniques. In one embodiment, a doctor may obtain a biopsy of the angiogenic region by inserting a bronchoscope through a subject's nose or mouth and down the throat to access the subject's airways and lungs and take a sample of the suspect tissue. Of course, alternative biopsy methods can be used. Biopsy techniques may be guided using aspects of the invention to make sure that a tissue sample containing abnormal vascular structures is removed. A suspect tissue sample can be analyzed in a laboratory, for example, to assay for the presence of one or more molecular indicators of cancer or other disease. However, in one embodiment, aspects of the invention provide a virtual biopsy that is sufficient to diagnose a condition without a tissue biopsy (e.g., a bronchoscopy biopsy). In one embodiment, aspects of the invention may be used to monitor a lesion (e.g. by analyzing it at several time points separated by relatively small time increments such as hours, days, or weeks) in order to determine whether it is growing and malignant, without involving an invasive biopsy procedure.

In one embodiment, subjects at risk of lung cancer may be screened routinely for abnormal lung vasculature structures according to aspects of the invention described herein. Risks of lung cancer include, but are not limited to, smoking, pollution, and family history.

Chronic Obstructive Pulmonary Disease (COPD). COPD is a term that is used for two closely related diseases of the respiratory system: chronic bronchitis and emphysema. In many patients these diseases occur together, although there may be more symptoms of one than the other.

In one embodiment, aspects of the invention may be used to detect early signs of COPD/Emphysema early and to monitor the progress of the disease and its response to drugs and other therapies. Early signs of COPD/Emphysema include increased blood vessel growth in diseased lungs in response to hypoxia. These signs may be detected before symptoms such as a chronic cough and progressive heart and lung failure develop. Subjects at risk, including smokers and subjects with mild shortness of breath, may be screened routinely according to methods of the invention.

Pulmonary Embolism (PE): Pulmonary embolism can result from a blocked artery in a subject's lung. Every year, more than 600,000 Americans experience a pulmonary embolism with severe and often fatal consequences. In most cases, the blockage is caused by one or more blood clots that had traveled to the lungs from another part of the body.

According to aspects of the invention, one or more blood clots may be detected before they travel to a subject's lungs and cause severe damage. The most common sources of blood clots are the deep veins of the leg. A clot may break loose from a leg vein and travel to a pulmonary artery in the lung, where it can block blood flow and cause more severe problems than when the clot was in the leg vein. Smaller clots prevent adequate blood flow to the lungs, sometimes causing damage to lung tissue (infarction). Large clots that completely block blood flow can be fatal. Aspects of the invention can be used to analyze leg vasculature to detect deep leg vein thrombosis. In people who receive treatment for deep leg vein thrombosis, the rate of pulmonary embolism falls to from a high of about 50% to less than 5%. Aspects of the invention also can be used to confirm the presence of deep leg vein thrombosis in patients who have symptoms such as leg pain or discomfort. It may be important to confirm the presence of deep leg vein thrombosis before administering an anticoagulant, because the treatment can cause adverse long-term complications.

Current techniques such as ventilation-perfusion scintigraphy, leg vein ultrasound, or pulmonary angiography are often not sufficient to establish a definitive diagnosis of pulmonary embolism or deep vein thrombosis. It should be apparent that aspects of the present invention may be used alone or in conjunction with current techniques to help detect and diagnose these conditions.

Aspects of the invention also may be useful to detect the full scope of blood vessel blockage in a subject's lung vasculature. Current techniques may detect certain blockages in large to medium sized pulmonary arteries (e.g., main, lobar and segmental). However, current techniques are of limited use for detecting blockages in sub-segmental and smaller blood vessels. Aspects of the invention may be used to detect patterns (e.g., individual structural features or distributions) indicative of blockages in these smaller blood vessels. This information can be used to optimize a subject's treatment.

Detection of lesions and/or disease locations: Lesions and/or disease locations may be detected by scanning an organ in full 3D and using disease specific vascular patterns as a way to detect the location and/or boundary of diseased tissue. By placing a 3D box around a suspicious area (e.g., one that was radiologically detected) and a disease specific vascular pattern may be used to detect the boundary of the diseased tissue.

Detection or Identification of Patients Most Likely to Respond to a Given Therapy:

Patients that are most likely to respond to a given therapy may be identified using a combination of moderately vascular diseased tissue along with the beginning of necrotic region(s) as a way to predict patients likely to respond to therapy (e.g., an anti-angiogenic therapy or an anti-cancer therapy). In addition, an increase in volume of a necrotic region of a patient identified above may be used as confirmation of a positive response to therapy.

Cancer/Angiogenesis:

Aspects of the invention may be used for tissue discrimination (e.g., for discriminating between normal and tumor tissue). In some embodiments, the presence of vessels alone may not be sufficiently informative and tissue and/or tumor-specific vascular patterns may be identified and used for analysis according to methods of the invention. In some embodiments, malignant and non-malignant soft tissue may be distinguished from each other (e.g., a benign cyst versus a tumor in a subject's breast; a benign versus a malignant lymph node in mediastinum). Parameters that may be used for discrimination may include, but are not limited to, one or more of the following: vascular diameter, vascular density (volume vessels/volume tumor), distribution curve of vascular diameters, inter-vessel distance, variability in vascular diameter, tortuosity, curvature, branching density, etc.

Aspects of the invention also may be used for therapeutic monitoring. This may involve quantification of one or more vasculature parameters. However, since the comparator is the same tumor or tissue prior to and after therapy, this monitoring may be accomplished without using specific patterns for identification of different tissues and/or tumors. In one embodiment, changes in vasculature pre- and post-therapy may be quantified (e.g., for previously identified, large (>1 cm) tumors in humans and large (>0.5 cm) tumors in mice). Parameters that may be used for therapeutic monitoring may include, but are not limited to, one or more of the following: vascular diameter, distribution of diameters, vascular density, inter-vessel distance, branching density, variability in vascular diameter (e.g., looking for “normalization”), tortuosity, curvature, etc. A therapeutic treatment may be evaluated on the basis of normalization (e.g., the score or quantitative measurement of the parameter returns towards a normal as opposed to a diseased level) of one or more of these parameters.

### Example 2

The following example relates to a particular model-based reconstruction technique for reconstructing images out of view data obtained from an x-ray or other scanning device. As described in detail below, such techniques can be employed to generate a model of small vasculature (e.g., blood vessels having a diameter of less than about 500 microns when using data from traditional CT scanners, or blood vessels having a diameter of less that about 50 microns when using data from X-ray scanners such as a micro-CT scanners that uses flat panel detectors). However, it should be appreciated that the techniques described below can be used to detect and reconstruct numerous other structures.

X-ray information about an object may be obtained by arranging an X-ray source and an array of detectors responsive to X-ray radiation about the object. Each detector in the array, for example, may generate an electrical signal proportional to the intensity of X-ray radiation impinging on a surface of the detector. The source and array may be rotated around the object in a circular path to obtain a number of views of the object at different angles. At each view, the detector signal generated by each detector in the array indicates the total absorption (i.e., attenuation) incurred by material substantially in a line between the X-ray source and the detector. Therefore, the array of detection signals records the projection of the object onto the detector array at a number of views of the object, and provides one method of obtaining view data of the object.

View data obtained from an X-ray scanning device may be of any form that provides attenuation information (e.g., detector outputs) as a function of view angle or orientation with respect to the object being imaged. View data may be obtained by exposing a planar cross-section of the object, referred to as a slice, to X-ray radiation. Each rotation about the object (e.g., a 180 degree rotation of the radiation source and detector array) provides attenuation information about a two-dimensional (2D) slice of the object.

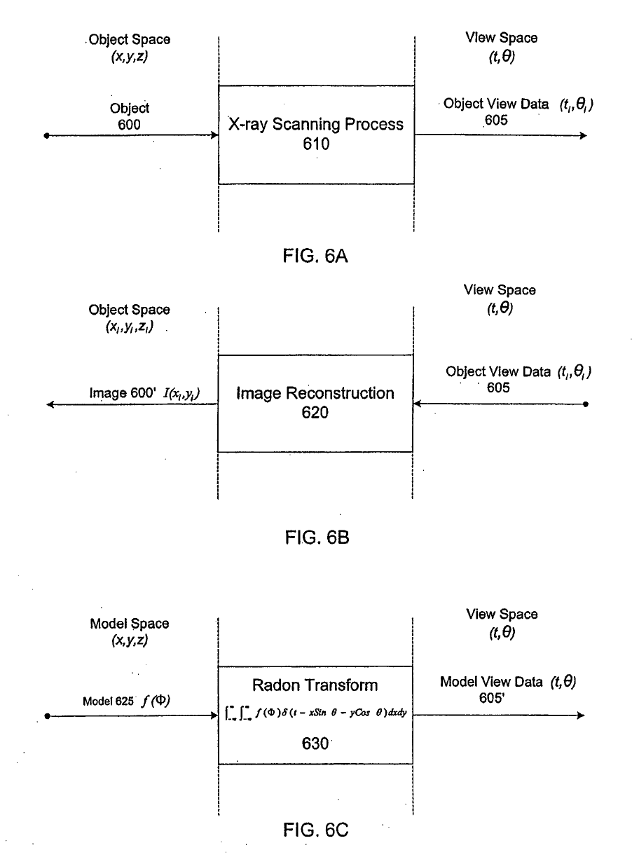

Accordingly, the X-ray scanning process transforms a generally unknown density distribution of an object into view data corresponding to the unknown density distribution. FIG. 6A illustrates a diagram of the transformation operation performed by the X-ray scanning process. A 2D cross-section of object 600 having an unknown density distribution in object space is subjected to X-ray scanning. Object space refers herein to the coordinate frame of an object of interest, for example, an object undergoing an X-ray scan. A Cartesian coordinate frame (i.e., (x, y, z)) may be a convenient coordinate system for object space, however, object space may be described by any other suitable coordinate frame, such as spherical or cylindrical coordinates.

X-ray scanning process 610 generates object view data 605 in a view space coordinate frame (e.g., coordinate frame (t,θ)). For example, object view data 605 may include attenuation information from a plurality of detectors in an array (corresponding to the view space axis tᵢ), at a number of orientations of the X-ray scanning device (corresponding to the view space axis θᵢ). Accordingly, X-ray scanning process 610 transforms a continuous density distribution in object space to discrete view data in view space.

To generate an image of the 2D density distribution from view data of an object, the view data may be projected back into object space. The process of transforming view data in view space into image data represented in object space is referred to as image reconstruction. FIG. 6B illustrates an image reconstruction process 620 that transforms view data 605 into a 2D image 600′ (e.g., a viewable image of the cross-section of object 600 that was scanned). To form 2D image 600′, a density value for each discrete location of the cross-section of object 600 in object space is determined based on the information available in view data 605. However, the process of image reconstruction results in a loss of resolution and detail in the reconstructed image.

Model-based imaging techniques have been employed to avoid some of the problems associated with loss of resolution and detail resulting from image reconstruction and to avoid segmentation difficulties posed by processing reconstructed images. Model-based techniques may include generating a model to describe structure assumed to be present in the view data of an object of interest. For example, a priori knowledge of the internal structure of an object of interest may be used to generate the model. The term “model” refers herein to any geometric, parametric or other mathematical description and/or definition of properties and/or characteristics of a structure, physical object, or system. For example, in an X-ray environment, a model of structure may include a mathematical description of the structure's shape and density distribution. A model may include one or more parameters that are allowed to vary over a range of values, such that the model may be deformed to take on a variety of configurations. The term “configuration” with respect to a model refers herein to an instance wherein each of the model parameters has been assigned a particular value.

Once a configuration of a model is determined, view data of the model (referred to as model view data) may be computed, for example, by taking the radon transform of the model. The radon transform, operating on a function, projects the function into view space. FIG. 6C illustrates the operation of the radon transform 630 on a model 625 of object 600. Model 625 is described by the function ƒ(Φ) in model space, where Φ is a vector of the parameters characterizing the model. Since model 625 is generated to describe object 600, it may be convenient to use the same coordinate frame for model space and object space, although they may be different so long as the transformation between the two coordinate frames are known. The radon transform 630 transforms model 625 from model space to model view data 605′ (i.e., to a function \tilde{g}ᵢ in the view space coordinate frame).

It should be appreciated that X-ray scanning process 610 and radon transform 630 perform substantially the same operation, i.e., both perform a transformation from object space (or model space) to view space. The scanning process performs a discrete transformation from object space to view space (i.e., to a discrete function in (θᵢ,tᵢ)) and the radon transform performs a continuous transformation from object space to view space (i.e., to a continuous function in (θ,t)). Model view data obtained by projecting a configuration of the model (i.e., an instance of ƒ where each parameter in Φ has been assigned a value) into view space via the radon transform, may then be compared to the object view data acquired from the X-ray scanning device to measure how accurately the model describes the structure of interest in the object being scanned. The model may then be deformed or otherwise updated until its radon transform (the model view data) satisfactorily fits the object view data, i.e., until the configuration of the model has been optimized. The optimization may be formulated, for example, by assuming that the observed object view data arose from structure that is parameterized as the model and finding the parameterization that best describes the object view data. For example, model deformation may be guided by minimizing the expression:

E(Φ)=∫_{N}(gᵢ(t,θ;Φ)−\tilde{g}ᵢ(t,θ;Φ))²dtdθ (1)

where Φ is a vector of the model parameters, gᵢ represents the object view data and \tilde{g}ᵢ represents the model view data. That is, the configuration of the model may be optimized by solving for the vector Φ that minimizes E (i.e., by finding the least squares distance).

Applicant has appreciated that when the structure being modeled is complex and includes a number of deformable parameters, the combinatorial problem of configuring the model may become intractable. That is, as the number of parameters over which the model is allowed to vary increases, the number of possible configurations of the model tends to explode. In addition, Applicant has appreciated that with no guidance on how to initially configure the model, a poorly chosen initial hypothesis may cause a subsequent optimization scheme to converge to an undesirable local minimum. As a result, the selected model configuration may poorly reflect the actual structure that was scanned.

Segmentation of reconstructed images is often difficult and is limited to information describing structure at the reduced resolution resulting from the image reconstruction process. Structure at or below this resolution, though present in the view data, may be unavailable to detection and segmentation algorithms that operate on reconstructed image data. Conventional model based techniques that seek to avoid image reconstruction have been frustrated by the combinatorial complexity of fitting a model configuration to the observed view data.

In one embodiment according to the present invention, a model is generated to describe structure to be detected in view data obtained from scanning the structure. The view data may be processed to detect one or more features in the view data characteristic of the modeled structure and employed to determine a value of one or more parameters of a configuration of the model, i.e., information in the view data may be used to bootstrap a hypothesis about how the model may be configured. By obtaining information about the model configuration from the view data, the combinatorial complexity of fitting the model configuration to observed view data and the likelihood of converging to an undesirable local minimum may be reduced. In addition, by processing the view data directly, structure may be detected at the resolution of the view data (i.e., substantially at the resolving capability of the X-ray scanning equipment.)

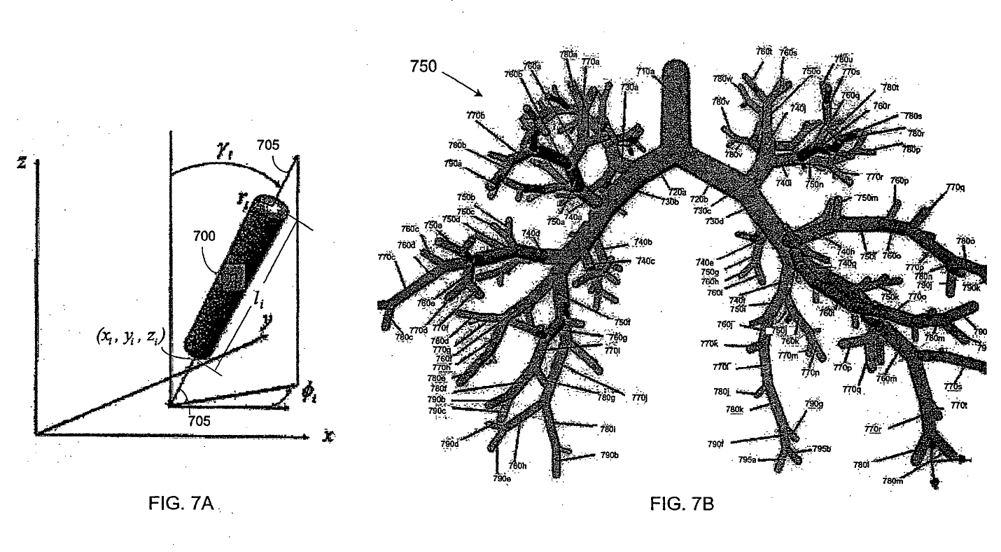

FIG. 7A illustrates one example of a cylindrical segment 700 that may be used as a component primitive in a cylinder network model. A configuration of cylindrical segment 700 may be described by a number of parameters in a particular coordinate frame (i.e., parameterized in model space). As discussed above, model space may be the same 3D coordinate frame as an object or structure being modeled (i.e., model space and object space may describe the same space). For example, the position of cylindrical segment 700 may be described by a location of the cylindrical axis 705 at a point (xᵢ, yᵢ, zᵢ) in space, for example, the origin or termination of the cylindrical segment. The orientation of cylindrical segment 700 may be specified by the angle øᵢ from the x-axis and the angle γᵢ from the y-axis. Since cylindrical segment 700 is axially symmetric, its rotation about the z-axis may not need to be specified. The length of the cylindrical segment may be specified by lᵢ and the radius of the cylindrical segment 700 may be specified by rᵢ. Accordingly, cylindrical segment 700 may be configured by assigning values to the seven parameters xᵢ, yᵢ, zᵢ, øᵢ, γᵢ, lᵢ and rᵢ.

FIG. 7B illustrates a configuration 750 of a cylindrical network model formed from a plurality of cylindrical segments arranged in a hierarchy. As discussed above, a vessel structure may include numerous vessels, each vessel having its own configuration in space to be described by the model. Configuration 750 includes a cylindrical segment 710a which branches into two cylindrical segments 720a and 720b, which further branch until the network terminates at the leaves of the hierarchy (i.e., cylindrical segments 720 branch into cylindrical segments 730, which in turn branch into segments 740, 750, 760 and so on). Although the specific parameter values are not shown, it should be appreciated that forming configuration 750 involves specifying values for the parameters of each of its component cylindrical segments. Modifying values of one or more of the parameters (including the number of cylindrical primitives in the hierarchy) results in a different model configuration.

It should be appreciated that the exemplary configuration 750 is a simplification of expected configurations for X-ray data with respect to the number of primitives in the configuration. In configuration 750 in example of FIG. 7B, configuration of the model involves specifying 7n parameters, where n is the number of cylindrical primitives. When all the parameters are unknown, optimization of configuration 750 involves several hundred degrees of freedom. A scanned portion of a vessel network may contain many times more vessels than described in configuration 750 (e.g., hundreds or thousands of vessels), making optimization of the configuration increasingly complex.

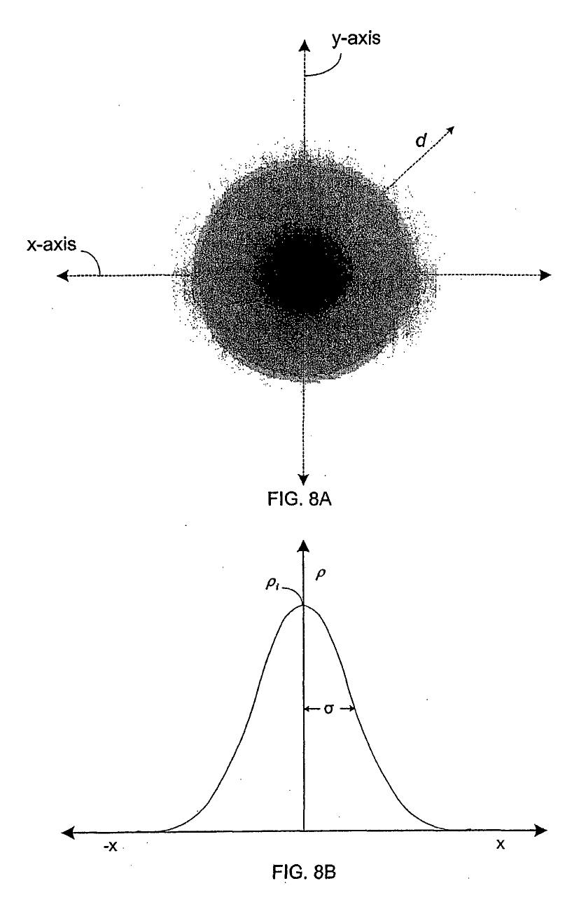

In one embodiment, the density distribution of the structure may also be modeled to understand how the structure projects into view space so that information gleaned therefrom can be used to assist in detecting features in view data corresponding to the modeled structure. For example, blood vessels may exhibit a characteristic density distribution that, when scanned, produces characteristic features or patterns in the view data. In one embodiment, the cross-sectional density of a vessel is modeled by a Gaussian distribution, centered on the longitudinal axis of the vessel, so that the modeled density is the highest at the center of the vessel. For example, the cross-sectional density distribution of cylindrical segment 700, when oriented such that its longitudinal axis coincides with the z-axis, may be modeled as,

$${ρ}_{i} {e}^{- \frac{1}{{r}_{i}^{2}} ( {( x - {x}_{i} )}^{2} + {( y - {y}_{i} )}^{2} )} \qquad ( 2 )$$

where ρᵢ is the density coefficient at a center of the i^{th} cylindrical segment and rᵢ is the radius of the i^{th} cylindrical segment, so that the density is modeled as being greatest at the center of the cylindrical segment (i.e., equal to ρᵢ) and decays exponentially as a function of radial distance from the center. FIG. 8A illustrates a grayscale representation of the function given in equation 2, where darker grayscale values indicate increased density values. FIG. 8B illustrates a plot of the intensity values along the x-axis at the center of the grayscale Gaussian distribution in FIG. 8A.

The density distribution along the longitudinal axis of the cylinder (i.e., into and out of the page in FIG. 8A) does not vary and may be modeled as a constant function of the cross-sectional distribution along the longitudinal axis, that is, as a constant function of the radial distance d from the center of the distribution. Accordingly, each cylindrical segment in configuration 750 may be assigned the cross-sectional density distribution defined in equation 2.

To express the density distribution at the orientation of a corresponding cylindrical segment, the density distribution may be transformed by the well known coordinate transformation matrix:

$$( \begin{matrix}cos [ γ ] cos [ φ ] & - sin [ φ ] & - cos [ φ ] sin [ γ ] \\ cos [ γ ] sin [ φ ] & cos [ φ ] & - sin [ γ ] sin [ φ ] \\ sin [ γ ] & 0 & cos [ γ ]\end{matrix} ) \qquad ( 3 )$$

where the angles γ and φ are the orientation parameters defined in FIG. 7A. It should be appreciated that the illustrative modeled density distribution of equation 2 depends only on the model parameters discussed in connection with FIG. 7A. Accordingly, if values have been assigned to each of the model parameters, the distribution may be fully described, such that the density distribution does not introduce any additional parameters. It should be appreciated that the invention is not limited in this respect, as the density distribution may be modeled such that it includes one or more independent model parameters.

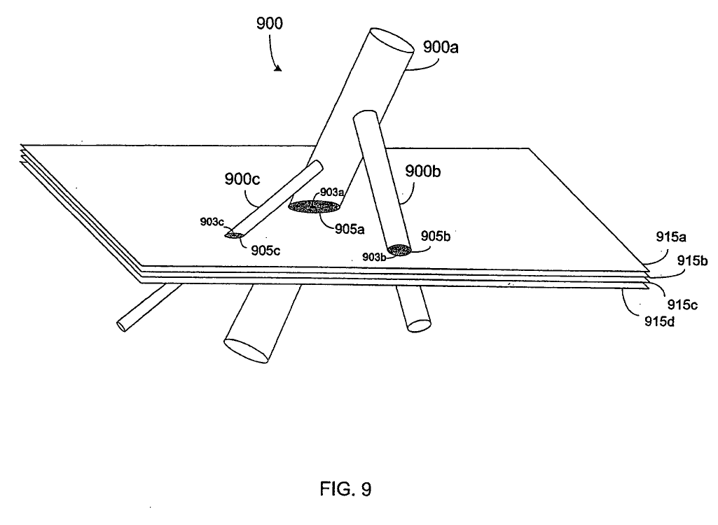

As discussed above, view data of a 3D object may be obtained by scanning a plurality of 2D cross-sections of the object. Applicant has recognized that detection of 3D structures of the object may be facilitated by considering how the structure appears when viewed at cross-sectional planes. For example, object 900 in FIG. 9 schematically represents a portion of a vessel network including vessels 900a, 900b and 900c. When object 900 is scanned, a plurality of cross-sectional slices of the object are exposed to X-ray radiation to provide view data corresponding to successive planes intersecting the object, e.g., exemplary planes 915a-915d.

The intersection of plane 915a with each of the cylindrical vessel segments produces respective ellipses 905a-905c, each having an eccentricity that depends on the angle the respective vessel segment cuts with the plane. Therefore, the presence of a 3D vessel segment may be detected by exploiting the recognition that from the perspective of an X-ray scanner, the vessel segments may appear as a succession of ellipses each having a characteristic density distribution (e.g., the density distribution described in equation 2).

It should be appreciated that when detecting features in 2D slices, the parameter zᵢ in FIG. 7A may be implied by the corresponding slice and therefore may not need to be determined to configure a cylindrical segment. In addition, in a scan plane, dimensions along the z-axis are infinitesimal. The appearance of the cylindrical segment in the plane is independent of the segment's length, so that the parameter lᵢ may go unspecified. Accordingly, in a 2D slice, a cross-section of a cylinder segment may be configured by assigning values to five parameters (i.e., xᵢ, yᵢ, øᵢ, γᵢ, and rᵢ).

To identify characteristic features in view data obtained from scanning vessel structures, a cross-section of a cylindrical primitive (i.e., an ellipse) having the density profile described in equation 2 may be projected into view space, e.g., by taking the radon transform of the density profile as discussed above. Accordingly, applying the density distribution in equation 2 to the general formulation of the radon transform gives,

$${\tilde{g}}_{i} ( t , θ ; Φ ) = {\int }_{- ∞}^{∞} {\int }_{- ∞}^{∞} {ρ}_{i} {e}^{- \frac{1}{{r}_{i}^{2}} ( {( x - {x}_{i} )}^{2} + {( y - {y}_{i} )}^{2} )} δ ( t - x Sin θ - y Cos θ ) d x d y \qquad ( 4 )$$

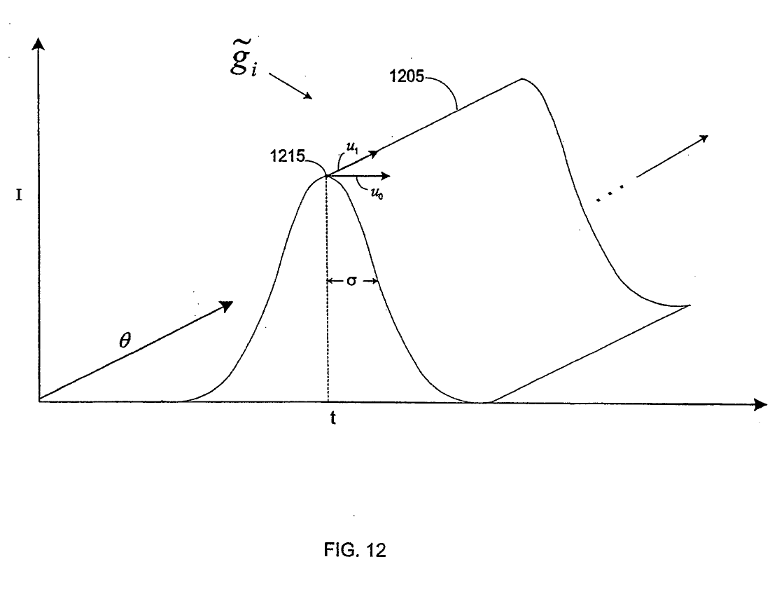

which results in the expression,

$${\tilde{g}}_{i} ( t , θ ; Φ ) = \sqrt{π} {ρ}_{i} {r}_{i} {e}^{- \frac{1}{{r}_{i}^{2}} ( {x}_{t} Sin θ + {y}_{t} Cos θ - t )} \qquad ( 5 )$$

where t and θ are axes of the coordinate frame in 2D view space and Φ represents the model parameters. Accordingly, when a blood vessel is scanned, it can be expected to give rise to information in the view data similar to the shape expressed in equation 5, which describes a sinusoidal function having a Gaussian profile. FIG. 12 illustrates schematically a segment of the function \tilde{g}ᵢ expressed in equation 5. Along the t-axis, \tilde{g}ᵢ has a characteristic Gaussian component. Along the θ-axis, \tilde{g}ᵢ has a characteristic sinusoidal component. As θ increases, the Gaussian component (i.e., the Gaussian profile along the t-axis) traces out a sinusoid.

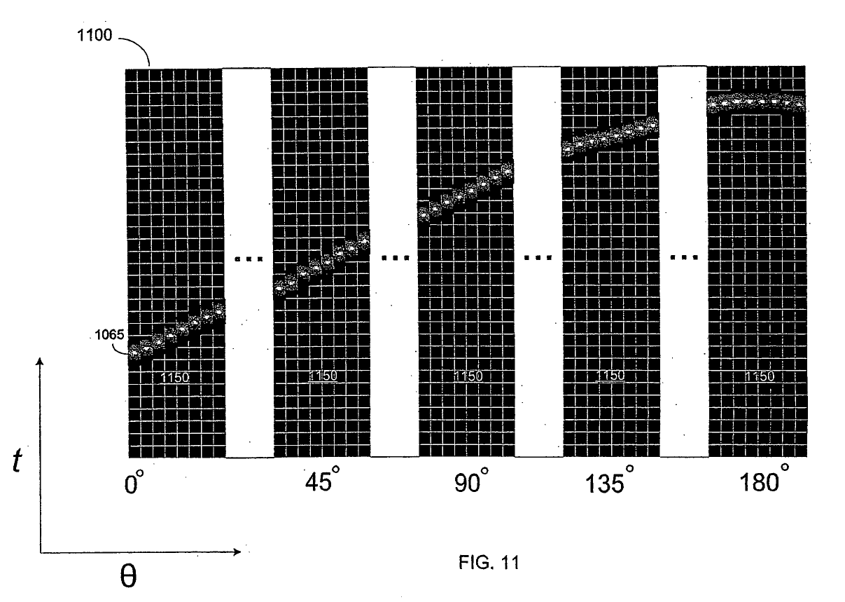

While only a short segment of the sinusoid (a small fraction of the period) is illustrated, it should be appreciated that peak 1215 of the Gaussian profile will trace out a sinusoid (better shown in FIG. 11) as indicated by sinusoidal segment 1205. As discussed below, this characteristic shape of the transformed Gaussian density distribution can be better understood by examining view data obtained from scanning an elliptical structure. In particular, scanning structure similar to the modeled cylindrical cross-section should produce discrete data that approximates the function of equation 5 due to the similar operations provided by the X-ray scanning process and the radon transform as discussed in connection with FIGS. 1A and 1C.

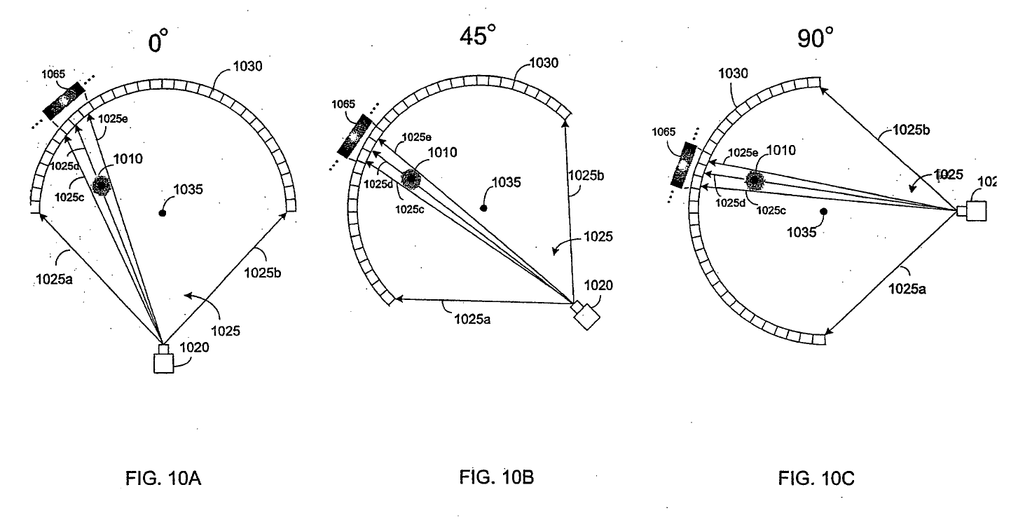

FIGS. 10A-10C illustrate a scanning operation of an ellipse 1010 having a Gaussian density distribution, such as shown in FIGS. 8A and 8B. For example, ellipse 1010 may be a cross-section of a vessel structure having a cross-sectional density similar to the density distribution in equation 2. The view data obtained from the scan is represented by sinogram 1100 illustrated schematically in FIG. 11. FIG. 10A illustrates a snapshot of portions of an X-ray scanning device 1000 at a 0° orientation, including a radiation source 1020 adapted to emit X-ray radiation and an array of detectors 1030 responsive to the X-ray radiation. Radiation source 1020 may emit a substantially continuous fan beam 1025, e.g., over an arc between rays 1025a and 1025b defining the extent of the fan beam. The radiation source 1020 may be positioned along the circular extensions of the semi-circular and detector adapted to rotate together with detector array 1030 about a center point 1035.

As the radiation source 1020 and the detector array 1030 rotate about center point 1035, the detectors in the array respond to impinging X-rays by generating a detection signal, for example, an electrical signal proportional to the intensity of the radiation impinging on respective detectors. As a result, the detector array records the radiation intensity profile at various orientations of the source and array with respect to ellipse 1010. The detection signals generated by each detector in the array may be sampled to obtain values indicating the intensity of an X-ray extending substantially in a line between each detector and the radiation source. The detector array may be sampled, for example, at a degree angle interval, half-degree angle interval, quarter-degree angle interval, etc., as the device rotates to obtain a number of projections of the ellipse at different views. FIGS. 10B and 10C illustrate snap-shots of the X-ray scanning device at 45° and 90°, respectively. A 2D scan of ellipse 1010 may include obtaining projections of ellipse 1010 over a 180° arc at a desired angle interval Δθ.

The majority of the radiation emitted by source 1020 will impinge unimpeded on the detector array 1030. However, some portion of the rays will pass through ellipse 1010 before reaching the detector array. The impeded rays will be attenuated to an extent related to the density of ellipse 1010. Exemplary rays 1025c and 1025e substantially tangent to the object will be the least attenuated rays of those that pass through the ellipse. Rays passing substantially through the center of ellipse 1010 (e.g., ray 1025d) have the most material to penetrate at the highest density and therefore will exhibit the greatest attenuation.

The detectors in the “shadow” of ellipse 1010, therefore, will detect radiation having a profile that transitions from zero attenuation at the tangent of ellipse 1010, to a peak attenuation at the center of ellipse 1010, and back to zero attenuation at the other tangent of ellipse 1010, as shown by profile 1065. For example, profile 1065 may be a grayscale representation of the detection signals provided by the detectors in the array that are in the shadow of the ellipse, wherein lighter gray levels indicate greater X-ray attenuation. Accordingly, detectors that are not in the shadow of ellipse 1010 produce detection signals having substantially black grayscale values. As expected from the Gaussian component of equation 5, i.e., the Gaussian profile illustrated in FIG. 12, profile 1065 has a characteristic Gaussian shape. That is, the Gaussian density distribution of ellipse 1010 projects Gaussian attenuation information onto the detector array.

Profile 1065 is illustrated at a higher resolution than the detector array, i.e., profile 1065 includes more than a single grayscale value for each detector in the shadow of ellipse 1010 to illustrate the characteristic Gaussian shape of the profile. However, it should be appreciated that each detector illustrated in detector array 1030 may be considered as any number of individual detectors generating detection signals such that a profile may be provided at the resolution of the illustrated profile 1065.

As the X-ray device rotates, the density distribution of the ellipse will project onto a changing combination of detectors. A 360° rotation of the device causes ellipse 1010 to orbit center point 1035 (from the perspective of radiation source 1020) causing the location of the ellipse projection on the detectors to repeat. As expected from the sinusoidal component of equation 5 (of which a segment is illustrated in FIG. 12) ellipse 1010 casts a periodic shadow that falls on the detectors at locations that trace across the detector array as a sinusoid as the orientation of the device increases, which can be mapped to 2D view space as discussed below.

FIG. 11 illustrates a sinogram 1100 of the view data obtained from scanning ellipse 1010 over a 1800 degree rotation at an angle interval of one degree. A sinogram is an image representation in view space of view data. In particular, a sinogram maps intensity values (e.g., attenuation values, density values, etc.) to a discrete coordinate location in view space. Sinogram 1100 has axes of θ and t, where θ represents the orientation of the X-ray device with respect to ellipse 1010 and t refers to a location along the detector array. Accordingly, sinogram 1100 provides a grayscale image of the detections signals generated by detector array 1030 as the X-ray scanning device rotates.

Specifically, sinogram 1100 includes a grid of pixels 1150, wherein each pixel has an intensity related to a sample of a detection signal from a respective detector in array 1030 at a particular orientation of the X-ray device. For example, the first column of pixels (θ=0), indicates samples from respective detectors responding to impinging radiation at a 0° orientation of the X-ray device. As a result, the characteristic profile 1065 from the detectors in the shadow of ellipse 1010, centered approximately at the ninth detector in the snapshot illustrated in FIG. 10A, appears centered approximately at pixel (0,9) in the sinogram. The second column of pixels indicates samples from respective detectors responding to impinging radiation at a 1° orientation of the X-ray device and so on at degree angle intervals.

As θ increases, the location of the profile 1065 traces out a portion of a sinusoid that reaches its half-period substantially at a 180° orientation. Portions of the sinogram 1100 are illustrated in the vicinity of a 45° orientation, a 90° orientation, a 135° orientation and a 180° orientation to illustrate the sinusoidal transition of the location of profile 1065 during the scan. It should be appreciated that the sinusoidal trace visible in sinogram 1100 provides a discrete approximation (represented as a grayscale image) of the function expressed in equation 5 (and illustrated in FIG. 12). Therefore, according to the model, a vessel structure that penetrates a particular scan plane or slice will generate a sinusoidal trace having a Gaussian profile in the sinogram associated with the slice. Detecting the presence of such characteristic sinusoids in the sinogram may indicate that the associated structure (e.g., a cross-section of a vessel) was present when the structure was scanned.

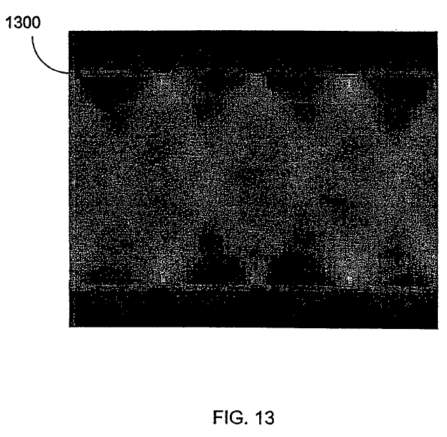

View data obtained from a scan of an object is likely to include sinusoidal traces from a variety of different structures (as opposed to the single trace in sinogram 1100). Projection information associated with the different structures may superimpose in view space. FIG. 13 illustrates a sinogram obtained from scanning an object having multiple unknown structures. Sinogram 1300 results from the superposition of numerous sinusoidal traces, some which may correspond to structure of interest and some which may not. To detect the structures of interest, features characteristic of the structure of interest may be distinguished from information corresponding to other structure and detected in the sinogram.

A Gaussian intensity distribution (e.g., the profile resulting from structure having a Gaussian density distribution) forms a ridge at the peak of the distribution. For example, peak 1205 in FIG. 12 forms a ridge that follows along the sinusoidal trace. Similarly, the lightest pixels in each of the Gaussian profiles 1065 (i.e., corresponding to the most attenuated X-rays) form a ridge point. Accordingly, ridge detection may be performed to identify characteristic features arising from a cross-section of the modeled vessel structure by locating ridge points in a sinogram formed from view data obtained from vessel structures.

A ridge point may be defined as a point in an image wherein the intensity assumes a local extrema in the direction of principal curvature, i.e., the direction having the steepest intensity gradient. For example, at point 1215 (and along peak 1205) in FIG. 12, the principal direction of curvature is shown by u₀ (i.e., the unit vector (1,0) in the (t, θ) coordinate frame). Each point along peak 1205 forms a ridge point since each point is so a local maximum along the t-axis (i.e., along the Gaussian profile). The term ridge is used herein to describe both local minimum and local maximum (i.e., to describe both crests and troughs having the above defined ridge characteristics).

A ridge may be characterized by local derivative information in the sinogram and may be detected by examining the curvature of intensity about points of interest in the sinogram. In one embodiment, a Hessian operator is used to extract curvature information from the sinogram to facilitate the detection of ridge points. In general terms, the purpose of applying the Hessian operator is to gather information concerning the way in which the intensity values vary in the pixels surrounding a pixel of interest. As discussed below, this information may be used to identify areas characteristic of a ridge. The Hessian operator in 2D may be expressed as,

$$H = [ \begin{matrix}\frac{{\partial }^{2} g}{\partial {t}^{2}} & \frac{{\partial }^{2} g}{\partial t \partial θ} \\ \frac{{\partial }^{2} g}{\partial t \partial θ} & \frac{{\partial }^{2} g}{\partial {θ}^{2}}\end{matrix} ] \qquad ( 6 )$$

where g is the sinogram operated on by the Hessian, and t and θ are the coordinate axes of the sinogram. For example, the Hessian operator may be applied to a sinogram by computing the Hessian matrix at each pixel or each of a subset of pixels in the sinogram, referred to as target pixels. The partial derivative elements of the Hessian matrix may be computed at each target pixel in a variety of ways. For example, the Hessian matrix may be determined by computing appropriate differences in a pixel neighborhood of the target pixel (e.g., an eight pixel adjacency neighborhood of the target pixel). Using a 3×3 neighborhood the Hessian matrix elements may be computed by weighting the pixel intensities according to corresponding elements of a discrete derivative mask and then summing the result. Exemplary derivative masks for the partial derivative elements of the Hessian are:

$$\frac{{\partial }^{2} g}{\partial {t}^{2}} = \frac{1}{3} [ \begin{matrix}1 & - 2 & 1 \\ 1 & - 2 & 1 \\ 1 & - 2 & 1\end{matrix} ] , \\ \frac{{\partial }^{2} g}{\partial {θ}^{2}} = \frac{1}{3} [ \begin{matrix}1 & 1 & 1 \\ - 2 & - 2 & - 2 \\ 1 & 1 & 1\end{matrix} ] , \\ and \\ \frac{{\partial }^{2} g}{\partial t \partial θ} = \frac{1}{4} [ \begin{matrix}1 & 0 & - 1 \\ 0 & 0 & 0 \\ - 1 & 0 & 1\end{matrix} ] . \qquad ( 7 )$$

The center of each matrix corresponds to the target pixel and the intensity of each of the eight adjacent pixels to the target pixel are multiplied by the corresponding element of the mask and summed together. The sum from each mask determines the corresponding element in the Hessian. It should be appreciated that other sized neighborhoods and different interpolating functions (i.e., the mask weights) for the pixels within the neighborhoods may be used, as the aspects of the invention relating to computing discrete partial derivatives are not limited to any particular method or implementation.

As discussed above, the Hessian describes the local curvature of intensity at pixels in the sinogram. The principal direction of curvature may be determined by decomposing the Hessian into its characteristic components. One method of determining the characteristic components of a matrix is to determine the eigenvalues and associated eigenvectors of the matrix.

In general terms, the eigenvectors of the Hessian matrix indicate the characteristic directions of curvature at a target pixel at which the Hessian was determined. As discussed below, the relationship between these characteristic directions of curvature may be employed to identify areas in the sinogram having characteristics of a ridge. The eigenvalues and associated eigenvectors of a matrix may be determined in various ways, for example, by any number of well known iterative methods of diagonalizing a matrix or analytically by directly solving the relationship:

Hu=λu (8)

where H is the Hessian matrix of equation 6, u is an eigenvector of matrix H, and λ is an eigenvalue associated with u. The magnitude of each eigenvalue of the Hessian is related to the “significance” of the associated eigenvector. Stated differently, the eigenvalue indicates how much the curvature along the associated eigenvector contributes to the local curvature determined by the Hessian. Accordingly, the largest eigenvalue of the Hessian matrix is associated with the principal direction of curvature.

As is well known, the 2D Hessian is a 2×2 symmetric matrix and therefore has two eigenvalues, λ₀ and λ₁, associated with respective and linearly independent eigenvectors u₀ and u₁ (i.e., eigenvectors u₀ and u₁ are orthogonal). The eigenvalue λ₀ herein denotes the eigenvalue having the greatest absolute value and is referred to as the principal eigenvalue. Accordingly, the associated eigenvector u₀ indicates the principal direction of curvature at a target pixel and λ₀ is related to the magnitude of the curvature. The eigenvalue λ₁ (referred to as the secondary eigenvalue) is related to the magnitude of curvature in the direction of u₁, i.e., in a direction orthogonal to the principal direction of curvature indicated by u₀.

At a ridge of a Gaussian profile of a sinusoidal trace, the curvature in a direction along the profile may be expected to be relatively large, while the curvature in an orthogonal direction along the ridge may be expected to be relatively small. Therefore, a ridge point may produce a large principal eigenvalue and a small secondary eigenvalue. For example, expected eigenvectors u₀ and u₁ are labeled at ridge point 1215 in FIG. 12. Since the curvature in the direction of u₀ is large, the magnitude of λ₀ is expected to be large as well. Likewise, since the intensity distribution is expected to be substantially uniform along the sinusoidal trace, the curvature in the direction of u₁ is theoretically zero and the magnitude of λ₁ is expected to be substantially zero. The values of and relationship between λ₀ and λ₁ may be employed to determine whether each target pixel at which the Hessian is computed is characteristic of a ridge point. That is, ridge points may have local curvature features expressed by the values of and/or the relationship between λ₀ and λ₁ that may be detected by evaluating the eigenvalues.

In one embodiment, a target pixel may be identified as a possible ridge point based on a predetermined criteria for the eigenvalues of the Hessian at the target pixel. For example, a threshold value may be applied to the magnitude of λ₀ to select as possible ridge points only target pixels having a principal eigenvalue that exceeds the threshold value. In addition or alternatively, a ratio of the magnitude of λ₀ to the magnitude of λ₁ may be subject to a threshold value such that ratios exceeding the threshold value are considered to have come from ridge points in the sinogram.

The sign of λ₀ may also be used to exclude ridges characterized by the wrong extrema (i.e., local minimum versus local maximum). For example, when the grey level scheme of the sinogram represents higher ray attenuation by lighter pixels (higher grey level values) as in FIG. 11, points giving rising to a negative λ₀ may be ignored (i.e., they indicate troughs rather than crests). Similarly, when the grey level scheme represents higher ray attenuation by lower grey level values, points giving rising to positive λ₀ may be ignored. Other criteria for evaluating eigenvalues and/or eigenvectors may be used, as aspects of the invention are not limited in this respect.

Accordingly, ridge detection may be applied to a sinogram to select ridge points by evaluating the local curvature characteristics about target points in the sinogram. The identified ridge points may indicate the presence of a Gaussian profile characteristic of a cross-section of a cylindrical structure (e.g., a blood vessel cross-section) in the corresponding slice of the object of interest. It should be appreciated that such ridge points derive their location in view space from the center location of the Gaussian density distribution, e.g., the center of a cross-section of a blood vessel. Accordingly, the location of the detected ridge points in a sinogram may be used to hypothesize the location of the center of a cylindrical segment at a cross-section corresponding to a slice from which the sinogram was obtained.

The detected ridge points may be transformed from view space (i.e., the coordinate frame (θ, t) of the sinogram) to model space (i.e., the coordinate frame (x, y, z) of the model) to determine a number of cylindrical primitives to use in the hypothesis and the location of the cylindrical axis of each of the cylindrical primitives. A sinusoidal trace characteristic of a vessel may generate numerous detected ridge points, for example, a sinusoid of ridge points that track substantially along the center of the trace (e.g., each of the lightest pixels in profile 1065 along the sinusoidal trace visible in sinogram 1100). However, many of the true ridge points of a particular sinusoidal trace may not be detected amongst other information in the sinogram corresponding to structure that occluded or partially occluded the vessel structure during the scan. Furthermore, as is often the case with thresholding techniques (e.g., the thresholds described above) some false positive ridge points may be detected.

Each ridge point that is part of the same sinusoidal trace is associated with the same ellipse center. Stated differently, each ridge point (θᵢ,tᵢ) in a same sinusoid in view space will transform to the same point (xᵢ,yᵢ) in model space. Since each characteristic sinusoidal trace is assumed to be generated by a corresponding vessel cross-section, the ridge point (i.e., the peak of the Gaussian distribution) corresponds to the center of the elliptical cross-section. Accordingly, the location of the cylindrical axis of a cylindrical segment where it intersects the scan plane corresponds to the transformed location of ridge points of the same sinusoidal trace.

The shape of a sinusoidal trace includes information about the location of corresponding structure in object space. For example, if ellipse 1010 in FIG. 10A-10C was positioned directly at center point 1035, the ellipse would generate a profile that traces a substantially horizontal line in the resulting sinogram (i.e., a sinusoidal trace having a zero amplitude) since the ellipse would cast a shadow on the same detectors independent of the orientation of the device. If the distance of ellipse 1010 from the center point 1035 were increased, the amplitude of the corresponding sinusoidal trace would also increase. The variation of the location of the profile is related to the distance of the ellipse from the center point 1035. Accordingly, the location of structure in object space (and thus model space) may be determined by examining the characteristics of the corresponding sinusoidal trace in view space in a manner discussed below.

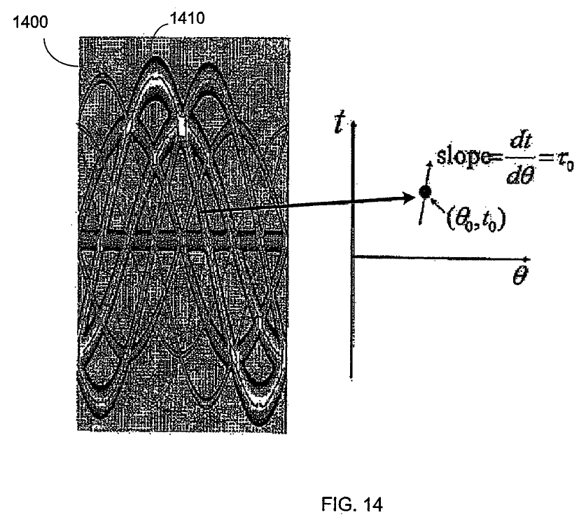

An example of determining object space locations from characteristics of a sinusoidal trace in view space will now be discussed, referring to the illustrative schematic sinogram 1400 shown in FIG. 14, which has a number of superimposed sinusoidal traces in view space resulting from unknown structure. Ridge detection may be applied to sinogram 1400 as discussed above to identify a pixel at (θ₀, t₀) as a ridge point of sinusoidal trace 1410. At the point (θ₀, t₀), the slope of the sinusoid 1410 is given by τ₀ and describes in part the shape of the sinusoidal trace. It is known from the radon transform that the sinusoidal trace in view space generated by a point (xᵢ, yᵢ) in object or model space, satisfies the expression:

xᵢ Sin θ+yᵢ Cos θ−t=0 (9).

To obtain two simultaneous equations, equation 9 may be differentiated with respect to θ, resulting in the expression:

$${x}_{i} Cos θ - {y}_{i} Sin θ - \frac{\partial t}{\partial θ} = 0. \qquad ( 10 )$$

By using the relationship

$$\frac{\partial t}{\partial θ} = τ$$

illustrated at (θ₀, t₀) in FIG. 14, τ may be substituted into equation 10, resulting in the expression:

xᵢ Cos θ−yᵢ Sin θ−τ=0 (11).

Since τ may be determined as discussed below, equations 9 and 11 provide two equations in two unknowns (i.e., xᵢ, yᵢ). Solving for the point (xᵢ, yᵢ) at the point (θ₀, t₀) results in the two expressions:

xᵢ=t₀ Sin θ₀+τ₀ Cos θ₀

yᵢ=t₀ Cos θ₀−τ₀ Sin θ₀ (12).

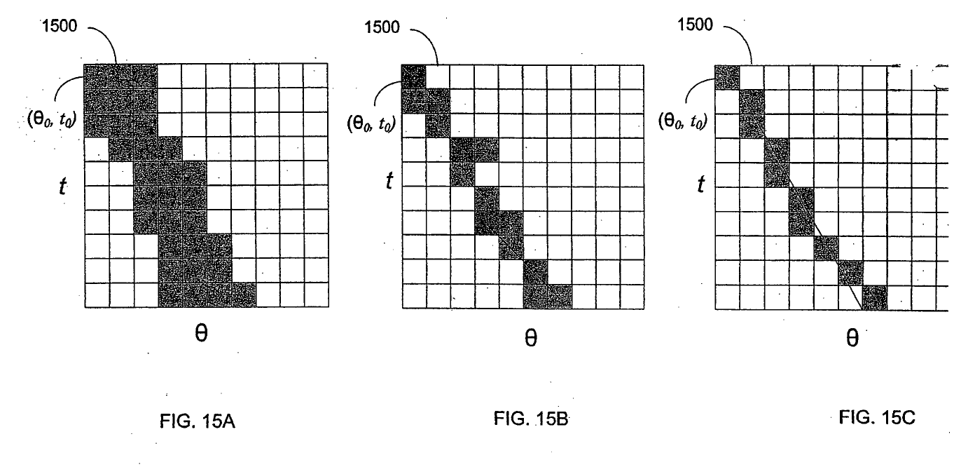

Accordingly, a point (θ₀, t₀) in view space may be transformed to a point (xᵢ, yᵢ) in object space if the slope of the sinusoidal trace τ₀ at (θ₀, t₀) is known or can be determined. The slope τ at a point (θ, t) may be computed in a variety of ways. For example, the slope τ may be computed by connecting adjacent detected ridge points to form a ridge segment. However, as discussed above, ridge detection may select a number of false ridge points that may frustrate attempts to connect detected ridge points into the correct ridge segments. Non-maximal suppression may be used to eliminate false ridge points as illustrated in FIGS. 15A-15C.

FIG. 15A illustrates a 10×10 pixel image portion 1500 of a sinogram. For example, image portion 1500 may be a portion of sinogram 1410 in the vicinity of point (θ₀, t₀). The shaded pixels denote points that were selected as possible ridge points during ridge detection. For example, each of the shaded pixels may have generated a Hessian having eigenvalues meeting some predetermined criteria. As discussed above, a ridge point is a local extrema in the direction of principal curvature. Accordingly, each pixel having an intensity that is not a local maximum may be eliminated. The shaded pixels in FIG. 15B illustrate local maxima computed with respect to the θ-axis.

When two adjacent pixels in the direction of non-maximum suppression have the same local maximum intensity, the pixel that generates the straightest line may be selected. For example, at the darker shaded pixels in FIG. 15B, more than one adjacent pixel could be chosen as belonging to the ridge segment. Pixels that form the straightest path (shown by the solid line segments) are selected over pixels that form the less direct paths (shown by the dotted lines). The shaded pixels in FIG. 15C illustrate the resulting ridge segment in local image portion 1500. The slope of the best fit line connecting the pixels in the ridge segment may be used as τ at each of the ridge points in the ridge segment (e.g., as τ₀ at ridge point (θ₀, t₀)).

The slope τ may also be computed individually at each target ridge point by taking the slope of the line connecting the selected ridge points in a local neighborhood of the target ridge point (e.g., estimating the slope by the connecting line through the target ridge point and the previous adjacent and subsequent adjacent ridge point). In detected ridge segments that are long, the local slope may provide a more accurate determination of the true slope of the sinusoidal trace at any given target ridge point. In FIGS. 15A-15C, non-maximal suppression was applied along the θ-axis. However, non-maximal suppression may be applied in any direction (e.g., in the direction of the principal eigenvector of the Hessian computed at the target ridge point). Alternatively, the slope τ may be determined at each ridge point according to the secondary eigenvector u₁. As shown in FIG. 12, eigenvector u₁ may point in a direction along the sinusoidal trace and may be used to estimate the slope τ in the transformation equations above.

As discussed above, each ridge point identified during ridge detection may be transformed to a coordinate location in model space. This transformed location corresponds to a hypothesized center of an elliptical cross-section, which in turn indicates the model space location where the axis of a cylindrical primitive intersects the plane of the associated slice (e.g., locations 903a-903c in FIG. 9). As discussed above, each ridge point belonging to a single sinusoidal trace should transform to the same coordinate location in model space. However, imprecision in computations (e.g., discrete partial derivative computations, tangent and/or slope computations, etc.), may cause particular transformed coordinates to deviate from the true model space location. However, transformed locations from multiple ridge points of the same sinusoidal trace can be expected to concentrate in a generally focused area. A location may be selected from this local concentration in any suitable way. For example, a histogram may be formed of locations transformed from each of the detected ridge points. Each ridge point effectively casts a vote for a location in model space.

In one embodiment, the histogram may be formed by discretizing model space into a grid. Each transformed ridge point may then be appropriately binned into the nearest location in the grid. Information in the resulting histogram may then be employed to determine both the number of cylindrical primitives to be used to configure the model, and the location of each primitive (i.e., the location of the longitudinal axis of the cylindrical primitive at an intersection with the plane of the corresponding slice). For example, a cylindrical primitive may be added to the cylinder network model for each local maximum or peak in the histogram. The cylindrical axis location of each added primitive may be initialized to correspond to the coordinate position in the grid corresponding to the peak in the histogram.

By determining the number of and location (i.e., parameters xᵢ, yᵢ) of cylindrical primitives that intersect a given slice, the combinatorial complexity of optimizing the model is significantly reduced. As discussed above, parameters for a cylindrical segment may include xᵢ, yᵢ, øᵢ, γᵢ, and rᵢ for each of the segments in the cylinder network model. The remaining model parameters in the configuration (e.g., øᵢ, γᵢ and rᵢ) for each of the determined primitives may be chosen in any suitable manner (e.g., based on a priori knowledge of the structure in the object of interest, by sampling a uniform distribution of values for each parameter, etc.). For example, the radii of the cylindrical primitives may be selected based on knowledge of the vessel size in the object or based on certain vessel sizes of particular interest.

Once the model has been configured, model view data of the model may be generated by taking the radon transform of the configured model. The model view data may then be compared with the object view data (i.e., the view data obtained from the X-ray scanning process) to obtain a measure of how well the configuration describes the structure that gave rise to the object view data. The configuration may then be updated until the model view data satisfactorily describes the object view data. Updating the configuration may be carried out using any suitable optimization technique. The optimized configuration may then be used as a description of the structure of interest in the object that was scanned.

In one embodiment, optimization of an initial model configuration may be further improved by determining values of one or more of the remaining parameters (e.g., radius and orientation for a cylinder) from observed view data for use in an initial hypothesis of the model configuration. In one embodiment discussed below, grayscale surface characteristics local to detected ridge points are employed to determine the radius of one or more of the cylindrical primitives comprising the cylinder network model. The grayscale distribution about the ridge may be analyzed to determine the radius of the associated structure. In particular, as a radius of a cylindrical cross-section is increased, so will the standard deviation of the Gaussian density distribution (i.e., the half-width of the Gaussian at the inflection point as shown by σ in FIG. 8B). As the variance of the Gaussian density distribution increases, so will the variance of the Gaussian profile component of the projection of the density distribution (i.e., the width of the Gaussian profile in the sinogram). The standard deviation (i.e., the square root of the variance) of the Gaussian may provide an approximation to the radius and may be expressed as,

$${r}_{i}^{2} = \frac{d g}{d t} {( \frac{{d}^{2} g}{d {t}^{2}} )}^{- 1} \qquad ( 13 )$$

where g is evaluated at the detected ridge points (i.e., equation 13 may be applied by taking the appropriate discrete derivatives of the intensity distribution about the ridge points). Other methods for evaluating the grayscale surface local to detected ridge points may also be used, as the aspect of the invention relating to determining an initial radius estimation is not limited to any particular implementation technique. For example, the distance to an inflection point of the intensity distribution in a direction along the principal direction of curvature (i.e., along the vector u₀) about each ridge point may be determined to estimate an initial value for the radius of each cylindrical segment in the model. Thus, in one embodiment, values for the radius parameter (i.e., rᵢ) may be determined from information in the view data.

The orientation of each cylindrical primitive may also be assigned one or more values based on information obtained from the sinogram to further improve the results and constrain the optimization. The orientation of the longitudinal axis of a cylindrical primitive is related to the eccentricity of the elliptical cross-section in a given slice. As shown in FIG. 9, the smaller the angle between the longitudinal axis of the cylinder and the plane of the slice, the greater the eccentricity. At one extreme, the cylindrical axis intersects the plane at a ninety degree angle resulting in an ellipse having an eccentricity of zero (i.e., a circle). At the other extreme, the cylindrical axis is parallel to the plane and a line having an eccentricity approaching infinity results. In one embodiment, the eccentricity may be computed from characteristics of the gray scale distribution in the sinogram using any suitable technique, to estimate an initial value for the orientation of each cylindrical segment in the model configuration.

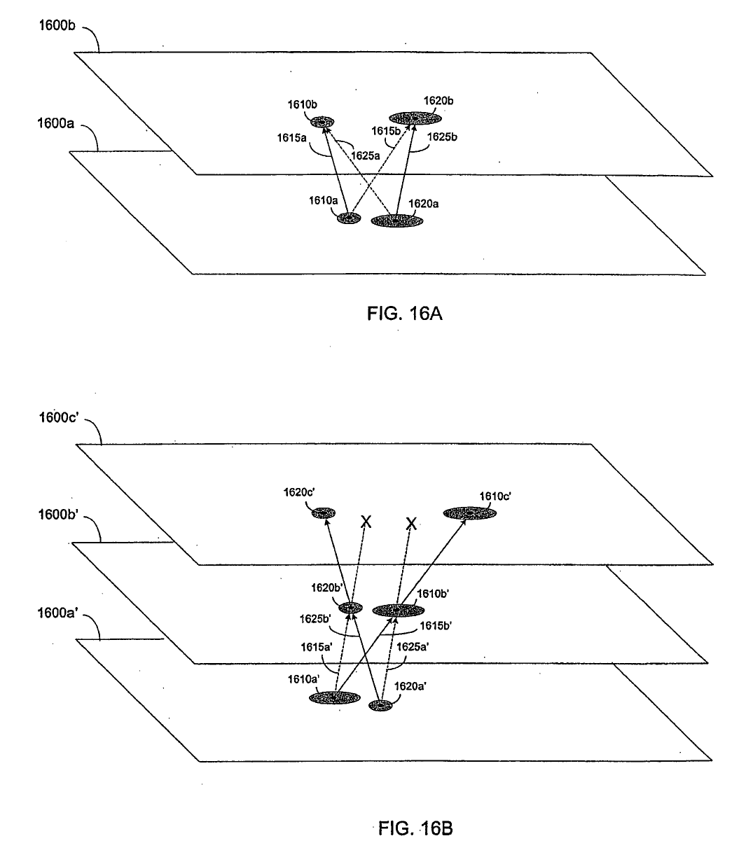

In another embodiment, information across multiple slices (i.e., 3D information) is used to determine cylinder axis orientation, in a manner described referring to FIGS. 16A and 16B. In FIG. 16A, locations 1610a and 1620a were detected as centers of elliptical cross-sections in a slice 1600a, e.g., by detecting and transforming ridge points in the sinogram of the slice. Similarly, locations 1610b and 1620b were detected as centers of elliptical cross-sections in another slice 1600b. When an ellipse center is detected in one slice, a corresponding ellipse center may be expected in nearby slices to account for the penetration of a cylindrical structure through multiple slices of the scan. The orientation of the cylindrical structure may be estimated from the change in location of the corresponding ellipse centers.

The orientation of each cylindrical primitive may be calculated by choosing a best fit between detected locations in successive slices. For example, a detected location having the shortest vector distance to a detected location in the subsequent slice may be determined to belong to the same cylindrical primitive. In FIG. 16A, location 1610a may be paired with location 1610b since no vector from location 1610a to any other detected location in slice 1600b has a magnitude less than the magnitude of vector 1615a. Similarly, location 1620a may be paired with 1620b. The direction of the vector connecting the paired locations may determine the orientation of the associated cylindrical primitive.

Using the shortest vector method in FIG. 16A, location 1610a′ may be incorrectly associated with location 1620b′ and location 1620a′ may be incorrectly associated with location 1610b′. To avoid this situation, in another embodiment described making reference to FIG. 16B, information in additional slices may be used. For example, the association between 1610a′ and 1620b′ may be checked against information in slice 1600c′. Since extensions of vectors 1615a′ and 1625a′ lead to locations where no ellipse centers were detected, the assumption made in the first instance may be penalized to prefer a global best fit, e.g., the grouping of locations 1610a′-1610c′ and the grouping of locations 1620a′-1620c′.

The detected locations in any number of slices may be analyzed together to determine the orientation of the various cylindrical primitives in the model configuration. For example, information in a group of N slices may be considered together, e.g., to constrain an optimization, regression and/or statistical scheme to determine the best fit groupings of the detected locations in the N slices. By tracking elliptical cross-section through the various slices, it may be determined when a particular cylinder first appears in a slice and when it terminates. This information may be used to determine the length lᵢ of each cylindrical segment. The orientation of the cylindrical primitives may be determined from the observed view data to facilitate a more accurate hypothesis of the initial configuration of the model.

It should be appreciated that one or any combination of model parameters of the cylindrical segment in FIG. 7A (i.e., xᵢ, yᵢ, øᵢ, γᵢ and rᵢ) may be configured based on information obtained from the view data, thus increasing the likelihood that the initial configuration is in the vicinity of the underlying structure and that subsequent optimization will converge to a close approximation of the modeled structure.

It should be appreciated that the view data operated on in methods of the various embodiments described herein may be at the maximum resolution that a given X-ray scanning device can generate. For example, various factors such as the number of detectors in the X-ray scanning device (or the sampling rate of a detector array), the angle interval over which the data is obtained, etc., limit the resolution of the view data. As discussed above, the resolution of the view data exceeds the resolution of images reconstructed from the data. For example, the resolution of the view data may be up to five times the resolution of the reconstructed image data, or more. Accordingly, by operating directly on the view data, various aspects of the invention may facilitate detection of structure at a higher resolution than available by detection methods applied to conventional reconstructed images.

Each of the different aspects, embodiments, or acts of the present invention described herein can be independently implemented in any of numerous ways. For example, each aspect, embodiments, or act can be independently implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments of the present invention comprises at least one computer-readable medium (e.g., a computer memory, a floppy disk, a compact disk, a tape, etc.) encoded with a computer program (i.e., a plurality of instructions), which, when executed on a processor, performs one or more of the above-discussed functions of the present invention. The computer-readable medium can be transportable such that the program stored thereon can be loaded onto any computer system resource to implement one or more functions of the present invention discussed herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

It should be appreciated that in accordance with several embodiments of the present invention wherein processes are implemented in a computer readable medium, the computer implemented processes may, during the course of their execution, receive input manually (e.g., from a user).

Having thus described several aspects of at least one embodiment of this invention, it is to be appreciated various alterations, modifications, and improvements will readily occur to those skilled in the art. Such alterations, modifications, and improvements are intended to be within the spirit and scope of the invention. Accordingly, the foregoing description and drawings are by way of example only.

What is claimed is:

1. A computer-implemented method of automatically analyzing in situ micro-vessels in an animal, the method comprising:
determining at least one structural parameter associated with in situ micro-vasculature in an animal, and,
determining whether said structural parameter is associated with at least one physiological indicium.

2. The method of claim 1, wherein said micro-vasculature comprises a micro-vessel with a diameter of less than 1 mm and said animal is a human.

3. The method of claim A1, wherein said micro-vasculature comprises a micro-vessel with a diameter of less than 200 microns and said animal is a small mammal and said small mammal is a rabbit, a mouse, a rat, or other small mammal.

4. The method of claim 1, wherein said physiological indicium is used for disease detection, disease diagnosis, disease staging, or therapy monitoring.

5. The method of claim 4, wherein said disease is a cancer.

6-22. (canceled)

23. A method of determining the presence or absence of a disease or one or more indicia of disease in a subject comprising computer-implemented or automated acts of:
analyzing at least one in situ tubular structure in a subject; and
determining from the in situ tubular structure analysis the presence or absence of a disease or one or more indicia of the disease in the subject.

24. The method of claim 23, wherein said at least one in situ tubular structure is a three-dimensional vascular structure.

25. The method of claim 24, wherein at least one structural parameter of the vascular structure is determined, and wherein said at least one structural parameter is vessel tortuosity, vessel branching, vessel diameter, vessel tree branch length, variability in vessel diameter, vessel curvature, branching density of said micro-vessel, vascular density in a target volume, vascular branching density in a target volume, micro-vessel diameter distribution within a target volume, or a combination thereof.

26. The method of claim 1, further comprising generating a score that indicates the probability that the in situ vasculature structure is associated with the disease.

27. The method of claim 26, wherein said score is generated by comparing said at least one in situ vasculature structure to a known structural parameter characteristic of a non-diseased vasculature.

28. The method of claim 27, further comprising comparing said score to a reference score.

29. The method of claim 23, wherein said structural parameter is detected using data obtained from a CT scan, a spiral CT scan, an MRI, a rotational digital X-ray scan, a scan using a rotational X-ray scanner having one or more flat panel detectors, a scan using a Tomosynthesis scanner having one or more rows of detector elements, a PET scan, a functional MRI, or a CT scanner having one or more rows of detector elements or a combination thereof.

30. (canceled)

31. A method of evaluating angiogenesis in a live subject, the method comprising computer-implemented acts of:
obtaining for a live subject a segmented representation of at least one in situ vasculature structure; and
gleaning from the in situ vasculature structure the presence or absence of angiogenesis in the live subject.

32. The method of claim 31, wherein said at least one in situ vasculature structure is a three-dimensional structure.

33. The method of claim 31, wherein at least one structural parameter of the vasculature structure is determined, and wherein said at least one structural parameter is blood vessel tortuosity, blood vessel branching, blood vessel diameter, blood vessel spatial distribution, or a combination thereof.

34. The method of claim 31, wherein at least one structural parameter is identified for a plurality of in situ blood vessels, and wherein said at least one in situ vasculature structure is a tissue density of said plurality of in situ blood vessels.

35. The method of claim 31, further comprising generating a score that indicates the probability that the in situ vasculature structure is associated with angiogenesis.

36. The method of claim 35, wherein said score is generated by comparing said at least one in situ vasculature structure to a known structural parameter characteristic of a non-angiogenic vasculature.

37. The method of claim 35, wherein said score is generated by quantifying said at least one in situ vasculature structure.

38. The method of claim 37, further comprising comparing said score to a reference score.

39-87. (canceled)
